SEQUENCE LISTING (6 sequences)

SEQ ID NO: 1 - DNA, 22 bp, Artificial Sequence
  Synthetic
gaccratcctgtcacctctgac

SEQ ID NO: 2 - DNA, 24 bp, Artificial Sequence
  Synthetic
agggcattctggacaaatcgtcta

SEQ ID NO: 3 - DNA, 24 bp, Artificial Sequence
  Synthetic
  misc_feature (1)..(1): Modified by FAM
  misc_feature (24)..(24): Modified by BHQ1
tgcagtcctcgctcactgggcacg

SEQ ID NO: 4 - DNA, 151 bp, Artificial Sequence
  Synthetic
gtctaatacgactcactataggaccaatcctgtcacctctgactgcagtcctcgctcactgggcacgtgcttcatcgcgaactgcttcgcggatgccatcgtcatggccacgaggatatgtaagagttagacgatttgtccagaatgccct

SEQ ID NO: 5 - DNA, 641 bp, Artificial Sequence
  Synthetic
actagaggagctactgtagtaattggaacaagcaaattctatggtggttggcacaacatgtattcaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatgattcgcacggtacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtc

SEQ ID NO: 6 - DNA, 477 bp, Artificial Sequence
  Synthetic
atgttaccttcttcatctacaataaaattgttgatgagcctgaagaacatgtccaaattgtaccaatcgacggttcatccggagttgttaatccagtaatggaagattcgcacggtccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaa

(19) United States
(12) Patent Application Publication
(10) Pub. No.: US 2023/0212701 A1
(43) Pub. Date: Jul. 6, 2023

(54) SYSTEMS AND METHODS FOR DETERMINING VIRUSES OR OTHER PATHOGENS

(71) Applicants: President and Fellows of Harvard College, Cambridge, MA (US); Montana State University, Bozeman, MT (US)

(72) Inventors: David A. Weitz, Cambridge, MA (US); Connie Chang Wilking, Bozeman, MT (US); Dimitri A. Bikos, Bozeman, MT (US); Emma Kate Loveday, Bozeman, MT (US); Geoffrey K. Zath, Bozeman, MT (US)

(73) Assignees: President and Fellows of Harvard College, Cambridge, MA (US); Montana State University, Bozeman, MT (US)

(21) Appl. No.: 17/922,284

(22) PCT Filed: Apr. 27, 2021
(86) PCT No.: PCT/US21/29423

§ 371 Date: Oct. 28, 2022

Related U.S. Application Data
(60) Provisional application No. 63/016,950, filed on Apr. 28, 2020.
(60) Provisional application No. 63/106,255, filed on Oct. 27, 2020.

(51) Int. Cl. C12Q 1/70

(52) U.S. Cl. CPC C12Q 1/701

(57) ABSTRACT

The present disclosure generally relates to systems and methods for detecting viruses, e.g., using microfluidic devices. Certain embodiments are generally directed to systems and methods that are able to detect pathogens such as viruses or bacteria by encapsulating a sample in droplets, and applying amplification reagents to the droplets able to amplify nucleic acids therein, e.g., using loop mediated isothermal amplification (LAMP) or other amplification techniques. In addition, some aspects are generally directed to identifying a species in a sample, e.g., at very low concentrations. In some cases, the sample may be broken into droplets, arid the droplets determined to determine the species.

### RELATED APPLICATIONS

This application claims the benefit of U.S. Provisional Patent Application Ser. No. 63/016,950, filed Apr. 28, 2020, entitled “High Throughput and High Sensitivity Detection of Disease Biomarkers and Pathogens,” by Wilking, et al. and U.S. Provisional Patent Application Ser. No. 63/106,255, filed Oct. 27, 2020, entitled “Systems and Methods for Determining Viruses or Other Pathogens,” by Weitz, et al. Each of these is incorporated herein by reference in its entirety.

### GOVERNMENT FUNDING

This invention was made with government support under contract W911NF-17-2-0034 awarded by the Defense Advanced Research Projects Agency (DARPA). The government has certain rights in the invention.

### FIELD

The present disclosure generally relates to systems and methods for detecting viruses, e.g., using microfluidic devices. In addition, certain embodiments relate to methods for high throughput and high sensitivity detection of nucleic acids of interest, namely for detection of disease and etiologic agents thereof.

### BACKGROUND

Coronaviruses are a group of viruses that cause diseases in mammals and birds. In humans, coronaviruses cause respiratory tract infections that are typically mild, such as the common cold, though rarer forms such as SARS, MERS and COVID-19 can be lethal. Coronaviruses are enveloped viruses with a positive-sense single-stranded RNA genome and a nucleocapsid of helical symmetry. The genome size of coronaviruses ranges from approximately 27 to 34 kilobases. The name coronavirus is derived from the Latin corona, meaning “crown” or “halo,” which refers to the characteristic appearance of the virus particles: they have a fringe reminiscent of a crown or of a solar corona.

### SUMMARY

The present disclosure generally relates to systems and methods for detecting viruses, e.g., using microfluidic devices. The subject matter of the present disclosure involves, in some cases, interrelated products, alternative solutions to a particular problem, and/or a plurality of different uses of one or more systems and/or articles.

For instance, one aspect is generally directed to producing droplets containing a sample within a microfluidic device, amplifying nucleic acid suspected of being in the droplets using loop mediated isothermal amplification, and determining fluorescence of the droplets.

Another aspect is generally directed to a method comprising mixing a saliva sample suspected of containing a target nucleic acid with an aqueous fluid to form a mixture, wherein the aqueous fluid comprises amplification reagents; forming a plurality of at least 10⁵ droplets of the mixture, contained within a carrier fluid; causing amplification of the target nucleic acid to produce amplified nucleic acids, using the amplification regents, within the plurality of droplets; determining droplets of the plurality of droplets that contain the amplified nucleic acids; and determining a nucleic acid concentration in the saliva sample based on the determination of the droplets containing the amplified nucleic acids.

Yet another aspect is generally directed to a method of determining an attomolar concentration of a species in a fluid. In one set of embodiments, the method comprises providing an aqueous fluid having a volume of at least 1 ml and containing a species at a concentration of less than 1 fM; forming a plurality of at least 10⁵ droplets of the aqueous fluid, contained within a carrier fluid; determining droplets of the plurality of droplets that contain the species; and determining a concentration of the species in the aqueous fluid based on the determination of droplets containing the species.

Still another aspect is generally directed to a method of detecting a species at an attomolar concentration. In some embodiments, the method comprises providing an aqueous fluid having a volume of at least 1 ml and containing a species at a concentration of less than 1 fM; forming a plurality of at least 10⁵ droplets of the aqueous fluid, contained within a carrier fluid; and determining droplets of the plurality of droplets that contain the species, wherein no more than 10 droplets per 10⁵ droplets contain the species.

Yet another aspect is generally directed to a method of detecting a target nucleic acid in saliva. In accordance with one set of embodiments, the method comprises mixing a saliva sample suspected of containing a target nucleic acid with an aqueous fluid to form a mixture, wherein the aqueous fluid comprises amplification reagents; forming a plurality of at least 10⁵ droplets of the mixture, contained within a carrier fluid; causing amplification of the target nucleic acid to produce amplified nucleic acids, using the amplification regent, within the plurality of droplets; and determining droplets of the plurality of droplets that contain the amplified nucleic acids, wherein no more than 10 droplets per 10⁵ droplets contain the amplified nucleic acids.

In another aspect, the present disclosure encompasses methods of making one or more of the embodiments described herein. In still another aspect, the present disclosure encompasses methods of using one or more of the embodiments described herein.

Other advantages and novel features of the present disclosure will become apparent from the following detailed description of various non-limiting embodiments of the disclosure when considered in conjunction with the accompanying figures.

### BRIEF DESCRIPTION OF THE DRAWINGS

Non-limiting embodiments of the present disclosure will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIGS. 1A-1B illustrate digital RT-LAMP detection and quantification, in one embodiment;

FIG. 2 illustrates dLAMP detection and quantification, in another embodiment;

FIG. 3 illustrates a device according to yet another embodiment;

FIGS. 4A-4E illustrate a device according to still another embodiment;

FIG. 5 illustrates an app in accordance with another embodiment;

FIG. 6 illustrates viral infection of Vero E6 cells;

FIGS. 7A-7C illustrate certain RT-LAMP primers, in yet another embodiment;

FIGS. 8A-8D illustrate various RT-LAMP prior sets and standard curves, in certain other embodiments;

FIGS. 9A-9B illustrate detection of genomic RNA using a primer set, in another embodiment;

FIGS. 10A-10C illustrate PCR thermal cycling, in certain other embodiments;

FIGS. 11A-11E illustrate a qRT-PCR dilution series, in another embodiment;

FIGS. 12A-12E illustrate amplification curves, in various embodiments;

FIGS. 13A-13E illustrate IAV amplification using qRT-PCR, in still another embodiment;

FIG. 14 illustrates droplets in a drop making device, in yet another embodiment;

FIG. 15 illustrates fluorescence for certain PCR conditions, in still other embodiments;

FIG. 16 illustrates amplification curves, in another embodiment;

FIGS. 17A-17B are various schematic diagrams of dPCR and RT-LAMP, in accordance with certain embodiments;

FIGS. 18A-18C illustrate certain ddPCR devices, in some embodiments; and

FIGS. 19A-19G illustrate detection of genes in drops, in yet other embodiments.

### DETAILED DESCRIPTION

The present disclosure generally relates to systems and methods for detecting viruses, e.g., using microfluidic devices. Certain embodiments are generally directed to systems and methods that are able to detect pathogens such as viruses or bacteria by encapsulating a sample in droplets, and applying amplification reagents to the droplets able to amplify nucleic acids therein, e.g., using loop mediated isothermal amplification (LAMP) or other amplification techniques. In addition, some aspects are generally directed to identifying a species in a sample, e.g., at very low concentrations. In some cases, the sample may be broken into droplets, and the droplets determined to determine the species.

In various aspects, the present disclosure relates to methods for the detection of molecules of interest with high throughput and high sensitivity. In one embodiment, the molecules of interest are biomolecules, which may include, but are not limited to, proteins, lipids, and nucleic acids. In a further embodiment, the biomolecules are known to be indicative of a current and/or past and/or future disease state. In a further embodiment, the disease state is attributed to the current and/or past presence of a pathogen. In a further embodiment, the pathogen is a bacteria, fungi, protozoan, and/or virus.

For example, one aspect of the present disclosure is generally directed to systems and methods for determining viruses, bacteria, or other pathogens. Examples include, but are not limited to, coronaviruses or other viruses such as those described herein. A sample, e.g., of a biological fluid taken from a subject, may be analyzed to determine whether a pathogen is present, e.g., a species of virus is present (e.g., SARS, MERS, COVID-19, etc.), and/or a type of virus is present (e.g., a coronavirus).

The sample of biological fluid may include fluids such as whole blood, blood serum, blood plasma, saliva, nasal fluid, sputum, urine, CNS fluid, breast nipple aspirate fluid, cerebral spinal fluid, semen, or the like. The subject that the biological fluid is taken from may be human, or non-human, e.g., a non-human mammal. Non-human mammals include, but are not limited to, a dog, cat, horse, cow, pig, sheep, goat, chicken, primate, rat, and mouse. In some cases, the subject is one that is suspected of being infected with a virus. For example, the subject may have previously been exposed to someone having the virus, or at least is suspected of potentially having the virus.

A variety of different viruses may be determined, in accordance with various embodiments. Non-limiting examples of viruses, including infectious viruses, include coronaviruses or influenza viruses. Other non-limiting examples include adenoviruses, coxsackieviruses, Epstein-Barr viruses, hepatitis viruses (A, B, and C), herpes simplex viruses (types 1 and 2), cytomegaloviruses, herpes viruses (type 8), HIV, measles viruses, mumps viruses, papilloma viruses, parainfluenza viruses, polioviruses, rabies viruses, respiratory syncytial viruses, rubella viruses, varicella-zoster viruses, etc.

In some embodiments, a coronavirus may be determined. Examples of coronaviruses include, but are not limited to, HCoV-229E, HCoV-OC43, SARS-CoV, HCoV-NL63, HKU1, MERS-CoV, or SARS-CoV-2. As discussed herein, in some cases, one or more proteins of the coronavirus may be used to determine the virus, e.g., by interaction with a binding moiety that is able to bind to the proteins, or a targeting species, such as are discussed herein. Examples of such proteins on viruses include, but are not limited to, peplomers, envelope proteins, membrane proteins, nucleocapsids, spike glycoproteins, hemagglutinin-esterase dimers (HE), or the like. In addition, in some cases, the nuclear material of the virus (for example, RNA) may be determined, e.g., by interaction with a binding moiety or a targeting species, etc.

In accordance with certain aspects, one example embodiment is generally directed to determining pathogens, such as viruses or bacteria, that may be present in a subject. This may be useful, for example, to quickly and accurately identify the pathogens such that the infection can be properly treated or managed, as delays can have significant mortality consequences. As an example, the subject may have or be at risk for a virus, e.g., a coronavirus such as COVID-19.

In some instances, a sample, such as a nasal or saliva sample, is analyzed to determine whether the pathogen is present. Accordingly, in this example, this process starts by acquiring a sample is taken from a subject. The sample may be optionally processed in some embodiments, e.g., cells within the sample may be lysed, or RNA may be purified, for example, by degrading the DNA using DNAses or other suitable procedures.

The sample may be encapsulated into droplets, e.g., for amplification. In some cases, the sample is encapsulated at relatively low concentrations, e.g., such that the droplets may, on the average contain less 1 nucleic acid molecule per droplet. This may be useful to ensure that most or all of the nucleic acid is amplified, e.g., substantially evenly.

In some cases, a plurality of primers may be present within the droplets to cause amplification. These may be, for example, RT-LAMP primers, droplet-based PCR primers, or other primers known to those of ordinary skill in the art, e.g., suitable for amplification.

After amplification, the amplified droplets may be determined or sequenced, e.g., using any of a variety of techniques. For instance, in one set of embodiments, the droplets may be broken and their contents optionally pooled together, e.g., to create a pool of amplified nucleic acids.

For example, in some cases, a species-selective primer targeting a virus (e.g., COVID-19) or other pathogen may be used to amplify nucleic acids arising from the pathogen, e.g., such that the pathogen can be identified as being present within the sample. After amplification, a variety of different techniques may be used to determine or identify the pathogen.

In some cases, the amplified nucleic acid may be contained within the droplets at a relatively low density, e.g., such that most or all of the droplets contain only a single type of nucleic acid or no nucleic acid. After amplification, the amplified nucleic acids may optionally be sequenced, e.g., using techniques such as those described herein, or otherwise analyzed. For instance, the presence and/or concentration of pathogens, such as viruses, present in a subject may be determined.

In certain aspects, techniques such as those described herein may be used to determine species such as viruses, bacteria, or other pathogens at relatively low concentrations. In some cases, surprisingly low concentration of a species may be determined, for example, species at concentrations of less than 1000 pM (picomolar), less than 100 pM, less than 10 pM, less than 1 pM, less than 100 fM (femtomolar), less than 10 fM, less than 1 fM, less than 100 aM (attomolar), less than 10 aM, or less than 1 aM.

It should be understood that such species are not limited to pathogens such as viruses, or bacteria. In other embodiments, other species may be determined, for example, species such as nucleic acids, particles such as microparticles or nanoparticles, colloidal particles, magnetic particles, quantum dots, fragrances, proteins, indicators, dyes, fluorescent species, chemicals, or the like.

In one set of embodiments, a sample suspected of containing such a species, e.g., at relatively low concentrations, may be determined as follows. The sample may be formed into a plurality of droplets. In some cases, relatively large numbers of droplets may be formed, e.g., at least about 50,000, at least about 100,000, at least about 300,000, at least about 1,000,000, at least about 3,000,000, at least 10,000,000, at least 100,000,00, or more droplets. The droplets may be determined to determine which of the droplets contains the species. It should be understood that, given sufficiently low concentrations, it is more proper to consider the species as being either present or not present within a droplet. Accordingly, determining the concentration of the species is equivalent to determining in which of the droplets the species is present in. Thus, for example, the species may be present in a relatively low number of the droplets, e.g., no more than 100, no more than 30, no more than 10, no more than 3, etc. of the droplets that are created from the sample may contain the species. Thus, by determining the droplets of the plurality of droplets that contain the species, the concentration of the species in the sample can be determined.

Any technique may be used to determine the species. For example, in one set of embodiments, such as discussed herein, a nucleic acid may be determined within a droplet by amplifying the droplet (e.g., using amplification reagents contained within the droplets), then detecting which droplets contain amplified nucleic acid, e.g., using fluorescence or other techniques such as those described herein. As another example, if the species of particles, the droplets can be determined to determine which droplets contain particles, e.g., if the particles are fluorescent, magnetic, etc. As still another example, an enzyme may be determined by determining a concentration of a substrate or product of the enzyme.

Accordingly, as mentioned, in certain aspects, RNA or other target nucleic acids arising from pathogens, such as viruses, may be present at a relatively low concentrations within a sample, such as a blood sample, which can be determined such as is discussed herein. Accordingly, some embodiments are generally directed to systems and methods of determining target nucleic acids in a sample, e.g., where the targets may be present at very low concentrations. For instance, a target nucleic acid may be present in a sample containing other nucleic acids at a concentration of 1:10³, 1:10⁴, 1:10⁵, 1:10⁶, 1:10⁷, 1:10⁸, 1:10⁹, or even lower concentrations.

Thus, in some cases, the nucleic acids may be amplified in some fashion as discussed above, e.g., to facilitate determination of the RNA or other target nucleic acids. For instance, the nucleic acids may be encapsulated into droplets. In some cases, the nucleic acids are encapsulated at relatively low concentrations, e.g., such that the droplets may, on the average contain less 1 nucleic acid per droplet. This may be useful to ensure that most or all of the nucleic acids are amplified, e.g., substantially evenly. In contrast, if the nucleic acids were to be amplified in bulk solution, some nucleic acids could be amplified without others being amplified (or being amplified to a much lesser degree). Thus, in certain embodiments as described herein the nucleic acids are encapsulated into droplets, and amplified therein.

In some cases, a plurality of primers may be added to the droplets to cause amplification, e.g., using loop mediated isothermal amplification, droplet-based PCR or other techniques known to those of ordinary skill in the art. In some cases, there may be at least 3, at least 5, at least 10, at least 30, at least 50, at least 100, at least 300, at least 500, at least 1,000, at least 2,000, at least 3,000, at least 5,000, or at least 10,000, or more distinguishable primers present. This may be useful, for example, to ensure a large number of potential target nucleic acids are amplified. However, in other embodiments, only a small number of primers may be present. For example, there may be less than 10, 9, 8, 7, 6, 5, 4, 3, 2, or only 1 primer that is added, e.g., to determine whether a particular pathogen, such as a virus (or group of viruses) is present.

In some cases, the amplified droplets may be determined or sequenced, e.g., using any of a variety of techniques. For instance, in one set of embodiments, the droplets may be broken and their contents pooled together, e.g., to create a pool of amplified nucleic acids. The pool of amplified nucleic acids may then be sequenced or determined (e.g., qualitatively or quantitatively), for example, using techniques such as Sanger sequencing, IIlumina sequencing, DNA microarrays, single-molecule real-time sequencing (e.g., Pacbio sequencing), nanopore sequencing, capillary electrophoresis, or the like. Determination of nucleic acids may include, as non-limiting examples, determining whether nucleic acid or a class of nucleic acids is present, determining some or all of the sequence of the nucleic acid, determining a concentration of the nucleic acid, etc. In some cases, the pool of amplified nucleic acids may be determined or identified, e.g., without any sequencing.

In addition, in certain embodiments, the pool of amplified nucleic acids may be sequenced using droplet-based techniques, e.g., droplet-based PCR. For example, in some cases, the amplified nucleic acids may be collected into droplets and the droplets exposed to certain primers, e.g., primers that are able to amplify rare target nucleic acid sequences. In some cases, the amplified nucleic acids may be collected into droplets at relatively low concentrations, e.g., such that the droplets may, on the average, contain less than 1 nucleic acid per droplet or less than 1 target per droplet, for instance, as described herein. In addition, in certain embodiments, the droplets may be divided into different groups of droplets, which are exposed to different primers. For instance, the droplets may be divided into at least 5, 10, 30, 100, etc. groups, which are exposed to various primers, e.g., in different spatial locations, to determine whether a target nucleic acid was present in the sample. However, it should be understood that in other embodiments, the amplified nucleic acids may be present at relatively higher concentrations, e.g., at at least 1 nucleic acid per droplet or at at least 1 target per droplet. In some cases, more than one primer or one amplicon may be present within a droplet.

A variety of target nucleic acids may be determined in accordance with various aspects. The nucleic acids may arise from a cell, such as a mammalian cell, or from other sources. The nucleic acids may be, for example, RNA and/or DNA, such as genomic DNA or mitochondrial DNA. In some cases, the nucleic acids are free-floating or contained within a fluid contained within the droplet. The nucleic acid may be taken from one or more cells (e.g., released upon lysis of one or more cells), synthetically produced, or the like. If the nucleic acid arises from cells, the cells may come from the same or different species (e.g., mouse, human, bacterial, etc.), and/or the same or different individual. For example, the nucleic acids may come from cells of a single organism, e.g., healthy or diseased cells (e.g., cancer cells), different organs of the organism, etc. In some cases, different organisms may be used (e.g., of the same or different species). In some cases, the nucleic acids may have a distribution such that some nucleic acids are not commonly present within a nucleic acid population. For example, there may be one cancer, disease cell, or pathogenic cell (e.g., a bacterium) among tens, hundreds, thousands, or more of normal or other cells. For instance, a subject may be infected with a pathogen, and identification of the pathogen may be desired, e.g., by determining its nucleic acids (e.g., DNA and/or RNA). Non-limiting examples of pathogens include bacteria, fungi, or the like. Specific examples include, but are not limited to, Staphylococcus aureus, Klebsiella pneumoniae, etc.

In one set of embodiments, RNA may be determined. For example, a sample, such as a blood sample, may be taken from a subject, and RNAs extracted from the sample. The sample may be any suitable sample. Examples of samples include, but are not limited to, cell culture fluid, water, saline, soil samples or other environmental samples, blood, or another bodily fluid, such as perspiration, saliva, plasma, tears, lymph, urine, plasma, or the like. In some cases, the fluid is an artificial fluid, e.g., cell culture fluid. In some cases, the fluid may arise, from a human or any other organism, e.g., a non-human mammal. In some cases, a sample of tissue, such as biopsy, may be taken and then homogenized or processed to separate cells, which may be used to form a suitable fluid, for instance, through admixture with saline. The fluid, in some embodiments, may be a relatively complex or biological mixture, e.g., containing a variety of cells and/or species, and in some cases, is not well-defined, e.g., unlike saline or a simple cell culture.

The cells within the sample may arise from any suitable source. For instance, the cells may be any cells for which nucleic acid from the cells is desired to be studied or sequenced, etc., and may include one, or more than one, cell type. The cells may be for example, from a specific population of cells, such as from a certain organ or tissue (e.g., cardiac cells, immune cells, muscle cells, cancer cells, etc.), cells from a specific individual or species (e.g., human cells, mouse cells, bacteria, etc.), cells from different organisms, cells from a naturally-occurring sample (e.g., pond water, soil, etc.), or the like. In some cases, the cells may be dissociated from tissue.

The subject may be, for example, a human subject. The subject may also be a non-human animal in some cases. Examples of such subjects include, but are not limited to, a mammal such as a cow, sheep, goat, horse, rabbit, pig, mouse, rat, dog, cat, a primate (e.g., a monkey, a chimpanzee, etc.), or the like. In some cases, the subject is a non-mammal such as a bird, an amphibian, or a fish.

Those of ordinary skill in the art will know of a variety of ways of extracting RNA from a sample, purifying and/or enriching a sample in RNA, etc. Certain non-limiting examples include those discussed in US Pat. Apl. Pub. No. 2019/0153427, incorporated herein by reference. As other examples, cells within a sample may be lysed, homogenized, etc., to release nucleic acids, such as DNA or RNA from the cells. For example, in some embodiments, cells within a sample may be lysed, for example, to release RNA and other contents from the cell, thereby producing a cell lysate. A variety of techniques can be used to lyse cells, such as exposure to a lysing chemical or a cell lysis reagent (e.g., a surfactant such as Triton-X or SDS, an enzyme such as lysozyme, lysostaphin, zymolase, cellulase, mutanolysin, glycanases, proteases, mannase, proteinase K, etc.), or a physical condition (e.g., ultrasound, ultraviolet light, mechanical agitation, etc.). Still other examples include chaotropic salts, detergents or alkaline denaturation.

Accordingly, in one set of embodiments, one or more cells may be lysed, and nucleic acids from the cells may be collected and distributed or encapsulated into droplets, e.g., as discussed herein. The lysing can be performed using any suitable technique for lysing cells. Non-limiting examples include ultrasound or exposure to suitable agents such as surfactants, or others such as those described herein. In some cases, the exact technique chosen may depend on the type of cell being lysed; many such cell lysing techniques will be known by those of ordinary skill in the art.

For example, in one set of embodiments, cells may be lysed by exposure to compounds such as enzymes (e.g., proteinases such as Proteinase K), lysozymes, EDTA (ethylenediaminetetraacetatic acid), surfactants (e.g., Tris-HCl), lysis buffers (e.g., Buffer RLT from Qiagen), guanidine isothiocycanates, beta-mercaptoethanol, or the like. Many such compounds can be readily obtained commercially. As another example, red blood cells may be lysed by exposing the red blood cells to compounds such as ammonium chloride, EDTA (ethylenediaminetetraacetatic acid), sodium bicarbonate, or the like. In addition, it should be understood that more than one method may be used to lyse cells. For example, a sample of blood may be exposed to an erythrocyte lysis buffer, one or more enzymes, and/or one or more mechanical techniques in order to lyse cells within the blood. If more than one technique is used, they may occur in any suitable order, and before, during, and/or after other techniques discussed herein.

In some cases, one or more RNA-stabilizing reagents may be added to the fluid to stabilize the RNA therein. The RNA-stabilizing reagent may be added to intact cells, or the cells within the fluid may be lysed prior to adding the RNA-stabilizing reagent. In some cases, the RNA-stabilizing reagent may be able to enter intact cells.

In one set of embodiments, the RNA-stabilizing reagent may include ammonium sulfate. The ammonium sulfate may be added, for example, such that the final concentration of ammonium sulfate in the fluid is no more than about 100 g/100 ml, no more than about 50 g/100 ml, no more than about 20 g/100 ml, no more than about 10 g/100 ml, or no more than about 5 g/100 ml. In some cases, the final concentration of ammonium sulfate may be no more than about 64 mM, no more than about 50 mM, no more than about 32 mM, no more than about 10 mM, no more than about 5 mM, etc. In addition, several RNA-stabilizing reagents may be obtained commercially, including RNAprotect Cell Reagent (Qiagen) or RNAlater (Thermo Fisher). Other examples of RNA-stabilizing reagents include those discussed in U.S. Pat. Apl. Pub. No. 2002/0115851.

In some cases, the RNA may be purified from the sample using techniques such as chemical-, column-, or gel-based approaches. Other approaches include organic phenol:chloroform extraction, binding of RNA to specific surfaces in the presence of chaotropic salts, exploiting RNA isolation on isopycnic gradients, or the like.

In certain embodiments, DNA from the sample may be degraded. In some cases, the DNA may be degraded using techniques that are selective for DNA, relative to RNA. In some cases, for instance, DNA may be degraded by exposing the lysate to a specific or a nonspecific endonuclease, e.g., one that preferentially acts on DNA relative to RNA. For example, the endonuclease may include a DNAse (a deoxyribonuclease) such as DNAse I, DNAse II, DNAse IV, UvrABC endonuclease, or the like. As another example, the DNA may be degraded via exposure to a restriction endonuclease. Many such nucleases are available commercially.

The sample may also be treated to separate nucleic acids in some cases. A variety of techniques can be used for such separation, including organic extraction (e.g., phenol, chloroform, and/or isoamyl alcohol), centrifugation, salting-out techniques (e.g., using potassium acetate or ammonium acetate), filtration, magnetic clearing, cesium chloride (CsCl) density gradients, solid-phase anion-exchange chromatography, binding to a solid-phase support (e.g., anion-exchange or silica), or the like. In one embodiment, for example, the lysate may be exposed to a nucleic acid separation column, e.g., containing silica. Many such columns are commercially available, and are typically used to separate DNA (not RNA) from cells. Non-nucleic acid species may be removed from the column, e.g., using suitable “wash” steps (e.g., using guanidine salts, ethanol, or the like), while nucleic acids (e.g., DNA and/or RNA) may subsequently be eluted from the column, e.g., using suitable elution buffers.

It should be noted that in some cases, there may be little DNA present, and/or the DNA that is present may be at least partially degraded, e.g., as discussed above, such that most of the nucleic acid eluted from the column is RNA.

In some aspects, a sample containing nucleic acids may be contained within a plurality of droplets, e.g., contained within a suitable carrying fluid. The nucleic acids may be present during formation of the droplets, and/or added to the droplets after formation. Any suitable method may be chosen to create droplets, and a wide variety of different droplet makers and techniques for forming droplets will be known to those of ordinary skill in the art. For example, a junction of channels may be used to create the droplets. The junction may be, for instance, a T-junction, a Y-junction, a channel-within-a-channel junction (e.g., in a coaxial arrangement, or comprising an inner channel and an outer channel surrounding at least a portion of the inner channel), a cross (or “X”) junction, a flow-focusing junction, or any other suitable junction for creating droplets. See, for example, International Patent Application No. PCT/US2004/010903, filed Apr. 9, 2004, entitled “Formation and Control of Fluidic Species,” by Link, et al., published as WO 2004/091763 on Oct. 28, 2004, or International Patent Application No. PCT/US2003/020542, filed Jun. 30, 2003, entitled “Method and Apparatus for Fluid Dispersion,” by Stone, et al., published as WO 2004/002627 on Jan. 8, 2004, each of which is incorporated herein by reference in its entirety.

In certain embodiments, nucleic acids may be added to droplet after the droplet has been formed, e.g., through picoinjection or other methods such as those discussed in Int. Pat. Apl. Pub. No. WO 2010/151776, entitled “Fluid Injection” (incorporated herein by reference), through fusion of the droplets with droplets containing the nucleic acids, or through other techniques known to those of ordinary skill in the art.

The nucleic acids may be contained within the droplets at relatively low densities, in accordance with certain embodiments. For instance, in one set of embodiments, the droplets may, on the average contain less 1 nucleic acid per droplet. For example, the average loading rate may be less than about 1 particle/droplet, less than about 0.9 nucleic acids/droplet, less than about 0.8 nucleic acids/droplet, less than about 0.7 nucleic acids/droplet, less than about 0.6 nucleic acids/droplet, less than about 0.5 nucleic acids/droplet, less than about 0.4 nucleic acids/droplet, less than about 0.3 nucleic acids/droplet, less than about 0.2 nucleic acids/droplet, less than about 0.1 nucleic acids/droplet, less than about 0.05 nucleic acids/droplet, less than about 0.03 nucleic acids/droplet, less than about 0.02 nucleic acids/droplet, or less than about 0.01 nucleic acids/droplet. In some cases, lower densities may be chosen to minimize the probability that a droplet will have two or more nucleic acids in it. Thus, for example, at least about 50%, at least about 60%, at least about 70%, at least about 80%, at least about 90%, at least about 95%, at least about 97%, at least about 98%, or at least about 99% of the droplets may contain either no target nucleic acid or only one such nucleic acid.

However, in some cases, the loading densities may also be controlled such that at least a significant amount of the droplets contains a target nucleic acid. This may be useful, for example, to prevent too much inefficiency in loading, or subsequent operations, etc. For instance, at least about 50%, at least about 60%, at least about 70%, at least about 80%, or at least about 90% of the droplets may also contain at least one such nucleic acid.

In some cases, the nucleic acids within the droplets may be amplified. This may be useful, for example, to produce a larger number or concentration of nucleic acids, e.g., for subsequent analysis, sequencing, or the like. Those of ordinary skill in the art will be familiar with various amplification methods that can be used, including, but are not limited to, polymerase chain reaction (PCR), reverse transcriptase (RT) PCR amplification, in vitro transcription amplification (IVT), multiple displacement amplification (MDA), quantitative real-time PCR (qPCR), loop mediated isothermal amplification (LAMP), or the like.

In some cases, the nucleic acids may be amplified within the droplets. This may allow amplification to occur “evenly” in some embodiments, e.g., such that the distribution of nucleic acids is not substantially changed after amplification, relative to before amplification. For example, according to certain embodiments, the nucleic acids within a plurality of droplets may be amplified such that the number of nucleic acid molecules for each type of nucleic acid may have a distribution such that, after amplification, no more than about 5%, no more than about 2%, or no more than about 1% of the nucleic acids have a number less than about 90% (or less than about 95%, or less than about 99%) and/or greater than about 110% (or greater than about 105%, or greater than about 101%) of the overall average number of amplified nucleic acid molecules per droplet. In some embodiments, the nucleic acids within the droplets may be amplified such that each of the nucleic acids that are amplified can be detected in the amplified nucleic acids, and in some cases, such that the mass ratio of the nucleic acid to the overall nucleic acid population changes by less than about 50%, less than about 25%, less than about 20%, less than about 15%, less than about 10%, or less than about 5% after amplification, relative to the mass ratio before amplification.

In some cases, certain primers are contained within the droplets to promote amplification. Such primers may be present during formation of the droplets, and/or added to the droplets after formation of the droplets. It should be noted that the manner in which the primers are added to the droplets may be the same or different from the manner in which the nucleic acids are added to the droplets.

In certain embodiments, a plurality of different types of primers may be added to the droplets. For instance, the primers may be distinguishable due to their having different sequences, and/or such that they are able to amplify different potential targets. In some cases, at least 2, at least 3, at least 5, at least 10, at least 15, at least 20, at least 25, at least 30, at least 40, at least 50, at least 60, at least 75, at least 100, at least 150, at least 200, at least 300, at least 400, at least 500, at least 1,000, at least 2,000, at least 3,000, at least 5,000, or at least 10,000, etc., different primers may be used. This may allow, for example, a variety of different target nucleic acids to be amplified within different droplets.

Examples of techniques for forming droplets include those described above. Examples of techniques for introducing primers after droplet formation include picoinjection or other methods such as those discussed in Int. Pat. Apl. Pub. No. WO 2010/151776, incorporated herein by reference, through fusion of the droplets with droplets containing primers, or the like. Other such techniques for either of these include, but are not limited to, any of those techniques described herein.

The primers may be present within the droplets at any suitable density. The density may be independent of the density of target nucleic acids. In some cases, an excess of primers are used, e.g., such that the target nucleic acids controls the reaction. For instance, if a large excess of primers are used, then substantially of the droplets will contain primer (regardless of whether or not the droplets also contain target nucleic acids). For example, in certain embodiments, at least about 50%, at least about 60%, at least about 70%, at least about 80%, at least about 90%, at least about 95%, at least about 97%, at least about 98%, or at least about 99% of the droplets may contain at least one amplification primer.

Droplets containing both primer and a target nucleic acid may be treated to cause amplification of the nucleic acid to occur. This may allow a large amount or concentration of the target nucleic acids to be produced, e.g., without substantially altering the distribution of nucleic acids. In some cases, the primers are selected to allow substantially all, or only some, of the target nucleic acids suspected of being present to be amplified.

As examples, PCR (polymerase chain reaction), LAMP, or other amplification techniques may be used to amplify nucleic acids, e.g., contained within droplets. For instance, in one embodiment, the amplification may be through loop mediated isothermal amplification (LAMP), using fluorescent detection. In LAMP, a nucleic acid sequence may be amplified using a relatively small number of primers (e.g., as discussed herein). In some cases, a polymerase with high strand displacement activity in addition to replication activity (e.g., Bst DNA polymerase, Bsm DNA polymerase, OmniAmp Pol, etc.) may be used. For example, 4 different primers may be used to amplify 6 distinct regions on the target gene, which increases specificity. An additional pair of “loop primers” can further accelerate the reaction. Thus, in some cases, a polymerase may be added to the droplets, e.g., in addition to primers such as discussed herein.

The amplification product can be determined, for example, via photometry, measuring the turbidity caused by magnesium pyrophosphate precipitate in solution as a byproduct of amplification. This allows visualization by the naked eye or via simple photometric detection approaches. The reaction can be followed in some cases in real-time, e.g., by measuring the turbidity, or by fluorescence, for example, using intercalating dyes such as SYTO 9. Various dyes, such as SYBR green, can be used to create a visible color change that can be seen with the naked eye, or determined by instrumentation, e.g., a spectrometer. In some cases, dye molecules may be able to intercalate or directly label the nucleic acids, and in turn can be correlated with the number of copies initially present. Hence, LAMP can also be quantitative in some embodiments.

In one set of embodiments, at least some of the primers may be distinguished, for example, using distinguishable fluorescent tags, barcodes, or other suitable identification tags. Examples of barcodes that can be contained within droplets include, but are not limited to, those described in U.S. Pat. Apl. Pub. No. 2018-0304222 or Int. Pat. Apl. Pub. No. WO 2015/164212, each incorporated herein by reference.

The nucleic acids may be amplified to any suitable extent. The degree of amplification may be controlled, for example, by controlling factors such as the temperature, cycle time, or amount of enzyme and/or deoxyribonucleotides contained within the droplets. For instance, in some embodiments, a population of droplets may have at least about 50,000, at least about 100,000, at least about 150,000, at least about 200,000, at least about 250,000, at least about 300,000, at least about 400,000, at least about 500,000, at least about 750,000, at least about 1,000,000 or more molecules of the amplified nucleic acid per droplet.

In one set of embodiments, the droplets are broken down after amplification, e.g., to allow the amplified nucleic acids to be pooled together. A wide variety of methods for “breaking” or “bursting” droplets are available to those of ordinary skill in the art. For example, droplets contained in a carrying fluid may be disrupted using techniques such as mechanical disruption, chemical disruption, or ultrasound. Droplets may also be disrupted using chemical agents or surfactants, for example, 1H,1H,2H,2H-perfluorooctanol.

After amplification, one or more of the nucleic acids may be determined or sequenced. However, it should be noted that because there are larger numbers of nucleic acids present, e.g., due to amplification, such analysis can be much easier. Such analysis can take many different forms in various embodiments, for instance, depending on factors such as the nature of the detection, the degree of quantification required, or the like.

Examples of methods for determining and/or sequencing nucleic acids include, but are not limited to, chain-termination sequencing, sequencing-by-hybridization, Maxam-Gilbert sequencing, dye-terminator sequencing, chain-termination methods, Massively Parallel Signature Sequencing (Lynx Therapeutics), polony sequencing, pyrosequencing, sequencing by ligation, ion semiconductor sequencing, DNA nanoball sequencing, single-molecule real-time sequencing (e.g., Pacbio sequencing), nanopore sequencing, Sanger sequencing, digital RNA sequencing (“digital RNA-seq”), Illumina sequencing, capillary electrophoresis, etc. In some cases, a microarray, such as a DNA microarray, may be used, for example to determine or identify nucleic acids. Those of ordinary skill in the art will be aware of other techniques that can be used to determine and/or sequence nucleic acids, e.g., qualitatively and/or quantitatively.

In addition, in some cases, the nucleic acids may be determined using droplet-based techniques, e.g., droplet-based PCR. As an example, the amplified nucleic acids may be contained within droplets, in accordance with certain embodiments, e.g., for subsequent analysis. The droplets may be created using any suitable technique, such as those described herein, and the technique for creating these droplets may be the same or different than for the initial droplets. In some cases, the droplets may also be monodisperse, and/or have distributions or dimensions such as are described herein. The amplified nucleic acids may be contained within droplets using any suitable technique, e.g., during or after the droplets have been formed. Techniques for creating droplets and/or adding fluid to a droplet have been discussed herein.

In some cases, the amplified nucleic acids may be contained within droplets at relatively low densities. For example, the droplets may, on the average contain less 1 nucleic acid per droplet. For example, the average loading rate may be less than about 1 nucleic acid/droplet, less than about 0.9 nucleic acids/droplet, less than about 0.8 nucleic acids/droplet, less than about 0.7 nucleic acids/droplet, less than about 0.6 nucleic acids/droplet, less than about 0.5 nucleic acids/droplet, less than about 0.4 nucleic acids/droplet, less than about 0.3 nucleic acids/droplet, less than about 0.2 nucleic acids/droplet, less than about 0.1 nucleic acids/droplet, less than about 0.05 nucleic acids/droplet, less than about 0.03 nucleic acids/droplet, less than about 0.02 nucleic acids/droplet, or less than about 0.01 nucleic acids/droplet. In some cases, lower densities may be chosen to minimize the probability that a droplet will have two or more nucleic acids in it. Thus, for example, at least about 50%, at least about 60%, at least about 70%, at least about 80%, at least about 90%, at least about 95%, at least about 97%, at least about 98%, or at least about 99% of the droplets may contain either no target nucleic acid or only one such nucleic acid. In addition, in some cases, the loading densities may also be controlled such that at least a signification amount of the droplets contains a target nucleic acid. This may be useful, for example, to prevent too much inefficiency in loading, or subsequent operations, etc. For instance, at least about 50%, at least about 60%, at least about 70%, at least about 80%, or at least about 90% of the droplets may also contain at least one such nucleic acid.

In some cases, the primers, if used, may be contained within the droplets using techniques such as those described herein. For instance, the primers may be present during formation of the droplets, and/or added to the droplets after formation of the droplets. It should be noted that the manner in which the primers are added to the droplets may be the same or different from the manner in which the nucleic acids are added to the droplets, and/or from the manner in which primers were added to the initial droplets.

In certain embodiments, the primers may be distributed such that some or all of the droplets contains only a single primer. For instance, at least about 50%, at least about 60%, at least about 70%, at least about 80%, at least about 90%, at least about 95%, at least about 97%, at least about 98%, or at least about 99% of the droplets may contain either no primer or only a single primer. In some cases, at least about 50%, at least about 60%, at least about 70%, at least about 80%, or at least about 90% of the droplets may contain only a single primer.

In one set of embodiments, at least some of the primers may be distinguished, for example, using distinguishable fluorescent tags, barcodes, or other suitable identification tags. Examples of barcodes that can be contained within droplets include, but are not limited to, those described in U.S. Pat. Apl. Pub. No. 2018-0304222 or Int. Pat. Apl. Pub. No. WO 2015/164212, each incorporated herein by reference.

In some embodiments, a plurality of different droplet makers may be used, each of which introduces a single primer into the droplets as they are formed. Examples of droplet makers include channel junctions such as a T-junction, a Y-junction, a channel-within-a-channel junction, a cross (or “X”) junction, a flow-focusing junction, or the like. Other suitable examples of different droplet makers and techniques for forming droplets include any of those discussed herein. Examples of techniques for introducing primers after droplet formation include picoinjection or other methods such as those discussed in Int. Pat. Apl. Pub. No. WO 2010/151776, incorporated herein by reference, through fusion of the droplets with droplets containing primers, or the like.

In some cases, the droplets may be divided into different groups such that the droplets are exposed to different primers, e.g., that are injected into the droplets. However, in other embodiments, the primers may be distributed differently, e.g., such that some or all of the droplets contains some or all of the primers.

Thus, in some embodiments, even though the primers may be distributed such that some or all of the droplets contains only a single primer in certain embodiments, because different groups of droplets are used, a plurality of different targets may still be determined for a pool of amplified nucleic acids. For instance, the droplets may be divided into at least 3, at least 5, at least 10, at least 30, at least 50, at least 100, at least 300, at least 500, at least 1,000, at least 2,000, at least 3,000, at least 5,000, or at least 10,000 or more groups, and some of the groups may be exposed to different primers, e.g., to determine if different target nucleic acids are present or not.

Droplets containing both primer and a nucleic acid may then be treated to cause amplification of the nucleic acid to occur, e.g., if the primer is one that can recognize the nucleic acid within the droplet and allow amplification to occur. In some embodiments, even relatively rare nucleic acids (e.g., having mutations) may be determined, for example, from a sample containing larger numbers of non-mutated nucleic acids. Techniques for amplifying nucleic acids include PCR (polymerase chain reaction) or any of the other techniques described herein.

After amplification, the amplified nucleic acids may optionally be determined and/or sequenced, e.g., using techniques such as those described herein. In some embodiments, the droplets may be burst and the nucleic acids may be combined to facilitate determination and/or sequencing, although in some cases, the determination and/or sequencing may occur within the droplets.

Examples of methods for determining and/or sequencing nucleic acids include, but are not limited to, chain-termination sequencing, sequencing-by-hybridization, Maxam-Gilbert sequencing, dye-terminator sequencing, chain-termination methods, Massively Parallel Signature Sequencing (Lynx Therapeutics), polony sequencing, pyrosequencing, sequencing by ligation, ion semiconductor sequencing, DNA nanoball sequencing, single-molecule real-time sequencing (e.g., Pacbio sequencing), nanopore sequencing, Sanger sequencing, digital RNA sequencing (“digital RNA-seq”), Illumina sequencing, etc. In some cases, a microarray, such as a DNA microarray, may be used, for example, to determine, or to sequence, a nucleic acid.

As mentioned, in some cases, the identity and/or the antibiotic susceptibility of the pathogens within the sample may be determined after determining and/or sequencing the species-specific and/or the gene-specific determinable nucleic acids within the sample, e.g., as was amplified as discussed herein. For example, the resistance of the pathogen to certain types of antibiotics (e.g., methicillin, ciprofloxacin, fluoroquinolone, carbapenem, etc.) may be determined, at least in part, based on the determination and/or sequencing of certain nucleic acids amplified using gene-specific primers.

Additional details regarding systems and methods for manipulating droplets in a microfluidic system follow, in accordance with certain aspects. For example, various systems and methods for screening and/or sorting droplets are described in U.S. patent application Ser. No. 11/360,845, filed Feb. 23, 2006, entitled “Electronic Control of Fluidic Species,” by Link, et al., published as U.S. Patent Application Publication No. 2007/000342 on Jan. 4, 2007, incorporated herein by reference. As a non-limiting example, in some embodiments, by applying (or removing) a first electric field (or a portion thereof), a droplet may be directed to a first region or channel; by applying (or removing) a second electric field to the device (or a portion thereof), the droplet may be directed to a second region or channel; by applying a third electric field to the device (or a portion thereof), the droplet may be directed to a third region or channel; etc., where the electric fields may differ in some way, for example, in intensity, direction, frequency, duration, etc.

As mentioned, certain embodiments comprise a droplet contained within a carrying fluid. For example, there may be a first phase forming droplets contained within a second phase, where the surface between the phases comprises one or more proteins. For example, the second phase may comprise oil or a hydrophobic fluid, while the first phase may comprise water or another hydrophilic fluid (or vice versa). It should be understood that a hydrophilic fluid is a fluid that is substantially miscible in water and does not show phase separation with water at equilibrium under ambient conditions (typically 25° C. and 1 atm). Examples of hydrophilic fluids include, but are not limited to, water and other aqueous solutions comprising water, such as cell or biological media, ethanol, salt solutions, saline, blood, etc. In some cases, the fluid is biocompatible.

Similarly, a hydrophobic fluid is one that is substantially immiscible in water and will show phase separation with water at equilibrium under ambient conditions. As previously discussed, the hydrophobic fluid is sometimes referred to by those of ordinary skill in the art as the “oil phase” or simply as an oil. Non-limiting examples of hydrophobic fluids include oils such as hydrocarbons oils, silicon oils, fluorocarbon oils, organic solvents, perfluorinated oils, perfluorocarbons such as perfluoropolyether, etc. Additional examples of potentially suitable hydrocarbons include, but are not limited to, light mineral oil (Sigma), kerosene (Fluka), hexadecane (Sigma), decane (Sigma), undecane (Sigma), dodecane (Sigma), octane (Sigma), cyclohexane (Sigma), hexane (Sigma), or the like. Non-limiting examples of potentially suitable silicone oils include 2 cst polydimethylsiloxane oil (Sigma). Non-limiting examples of fluorocarbon oils include FC3283 (3M), FC40 (3M), Krytox GPL (Dupont), etc. In addition, other hydrophobic entities may be contained within the hydrophobic fluid in some embodiments. Non-limiting examples of other hydrophobic entities include drugs, immunologic adjuvants, or the like.

Thus, the hydrophobic fluid may be present as a separate phase from the hydrophilic fluid. In some embodiments, the hydrophobic fluid may be present as a separate layer, although in other embodiments, the hydrophobic fluid may be present as individual fluidic droplets contained within a continuous hydrophilic fluid, e.g., suspended or dispersed within the hydrophilic fluid. This is often referred to as an oil/water emulsion. The droplets may be relatively monodisperse, or be present in a variety of different sizes, volumes, or average diameters. In some cases, the droplets may have an overall average diameter of less than about 1 mm, or other dimensions as discussed herein. In some cases, a surfactant may be used to stabilize the hydrophobic droplets within the hydrophilic liquid, for example, to prevent spontaneous coalescence of the droplets. Non-limiting examples of surfactants include those discussed in U.S. Pat. Apl. Pub. No. 2010/0105112, incorporated herein by reference. Other non-limiting examples of surfactants include Span80 (Sigma), Span80/Tween-20 (Sigma), Span80/Triton X-100 (Sigma), Abil EM90 (Degussa), Abil we09 (Degussa), polyglycerol polyricinoleate “PGPR90” (Danisco), Tween-85, 749 Fluid (Dow Corning), the ammonium carboxylate salt of Krytox 157 FSL (Dupont), the ammonium carboxylate salt of Krytox 157 FSM (Dupont), or the ammonium carboxylate salt of Krytox 157 FSH (Dupont). In addition, the surfactant may be, for example, a peptide surfactant, bovine serum albumin (BSA), or human serum albumin.

The droplets may have any suitable shape and/or size. In some cases, the droplets may be microfluidic, and/or have an average diameter of less than about 1 mm. For instance, the droplet may have an average diameter of less than about 1 mm, less than about 700 micrometers, less than about 500 micrometers, less than about 300 micrometers, less than about 100 micrometers, less than about 70 micrometers, less than about 50 micrometers, less than about 30 micrometers, less than about 10 micrometers, less than about 5 micrometers, less than about 3 micrometers, less than about 1 micrometer, etc. The average diameter may also be greater than about 1 micrometer, greater than about 3 micrometers, greater than about 5 micrometers, greater than about 7 micrometers, greater than about 10 micrometers, greater than about 30 micrometers, greater than about 50 micrometers, greater than about 70 micrometers, greater than about 100 micrometers, greater than about 300 micrometers, greater than about 500 micrometers, greater than about 700 micrometers, or greater than about 1 mm in some cases. Combinations of any of these are also possible; for example, the diameter of the droplet may be between about 1 mm and about 100 micrometers. The diameter of a droplet, in a non-spherical droplet, may be taken as the diameter of a perfect mathematical sphere having the same volume as the non-spherical droplet.

In some embodiments, the droplets may be of substantially the same shape and/or size (i.e., “monodisperse”), or of different shapes and/or sizes, depending on the particular application. In some cases, the droplets may have a homogenous distribution of cross-sectional diameters, i.e., in some embodiments, the droplets may have a distribution of average diameters such that no more than about 20%, no more than about 10%, or no more than about 5% of the droplets may have an average diameter greater than about 120% or less than about 80%, greater than about 115% or less than about 85%, greater than about 110% or less than about 90%, greater than about 105% or less than about 95%, greater than about 103% or less than about 97%, or greater than about 101% or less than about 99% of the average diameter of the microfluidic droplets. Some techniques for producing homogenous distributions of cross-sectional diameters of droplets are disclosed in International Patent Application No. PCT/US2004/010903, filed Apr. 9, 2004, entitled “Formation and Control of Fluidic Species,” by Link, et al., published as WO 2004/091763 on Oct. 28, 2004, incorporated herein by reference. In addition, in some instances, the coefficient of variation of the average diameter of the droplets may be less than or equal to about 20%, less than or equal to about 15%, less than or equal to about 10%, less than or equal to about 5%, less than or equal to about 3%, or less than or equal to about 1%. However, in other embodiments, the droplets may not necessarily be substantially monodisperse, and may instead exhibit a range of different diameters.

Those of ordinary skill in the art will be able to determine the average diameter of a population of droplets, for example, using laser light scattering or other known techniques. The droplets so formed can be spherical, or non-spherical in certain cases. The diameter of a droplet, in a non-spherical droplet, may be taken as the diameter of a perfect mathematical sphere having the same volume as the non-spherical droplet.

In some embodiments, one or more droplets may be created within a channel by creating an electric charge on a fluid surrounded by a liquid, which may cause the fluid to separate into individual droplets within the liquid. In some embodiments, an electric field may be applied to the fluid to cause droplet formation to occur. The fluid can be present as a series of individual charged and/or electrically inducible droplets within the liquid. Electric charge may be created in the fluid within the liquid using any suitable technique, for example, by placing the fluid within an electric field (which may be AC, DC, etc.), and/or causing a reaction to occur that causes the fluid to have an electric charge.

The electric field, in some embodiments, is generated from an electric field generator, i.e., a device or system able to create an electric field that can be applied to the fluid. The electric field generator may produce an AC field (i.e., one that varies periodically with respect to time, for example, sinusoidally, sawtooth, square, etc.), a DC field (i.e., one that is constant with respect to time), a pulsed field, etc. Techniques for producing a suitable electric field (which may be AC, DC, etc.) are known to those of ordinary skill in the art. For example, in one embodiment, an electric field is produced by applying voltage across a pair of electrodes, which may be positioned proximate a channel such that at least a portion of the electric field interacts with the channel. The electrodes can be fashioned from any suitable electrode material or materials known to those of ordinary skill in the art, including, but not limited to, silver, gold, copper, carbon, platinum, copper, tungsten, tin, cadmium, nickel, indium tin oxide (“ITO”), etc., as well as combinations thereof.

In another set of embodiments, droplets of fluid can be created from a fluid surrounded by a liquid within a channel by altering the channel dimensions in a manner that is able to induce the fluid to form individual droplets. The channel may, for example, be a channel that expands relative to the direction of flow, e.g., such that the fluid does not adhere to the channel walls and forms individual droplets instead, or a channel that narrows relative to the direction of flow, e.g., such that the fluid is forced to coalesce into individual droplets. In some cases, the channel dimensions may be altered with respect to time (for example, mechanically or electromechanically, pneumatically, etc.) in such a manner as to cause the formation of individual droplets to occur. For example, the channel may be mechanically contracted (“squeezed”) to cause droplet formation, or a fluid stream may be mechanically disrupted to cause droplet formation, for example, through the use of moving baffles, rotating blades, or the like.

Some embodiments generally relate to systems and methods for fusing or coalescing two or more droplets into one droplet, e.g., where the two or more droplets ordinarily are unable to fuse or coalesce, for example, due to composition, surface tension, droplet size, the presence or absence of surfactants, etc. In certain cases, the surface tension of the droplets, relative to the size of the droplets, may also prevent fusion or coalescence of the droplets from occurring.

As a non-limiting example, two droplets can be given opposite electric charges (i.e., positive and negative charges, not necessarily of the same magnitude), which can increase the electrical interaction of the two droplets such that fusion or coalescence of the droplets can occur due to their opposite electric charges. For instance, an electric field may be applied to the droplets, the droplets may be passed through a capacitor, a chemical reaction may cause the droplets to become charged, etc. The droplets, in some cases, may not be able to fuse even if a surfactant is applied to lower the surface tension of the droplets. However, if the droplets are electrically charged with opposite charges (which can be, but are not necessarily of, the same magnitude), the droplets may be able to fuse or coalesce. As another example, the droplets may not necessarily be given opposite electric charges (and, in some cases, may not be given any electric charge), and are fused through the use of dipoles induced in the droplets that causes the droplets to coalesce. Also, the two or more droplets allowed to coalesce are not necessarily required to meet “head-on.” Any angle of contact, so long as at least some fusion of the droplets initially occurs, is sufficient. See also, e.g., U.S. patent application Ser. No. 11/698,298, filed Jan. 24, 2007, entitled “Fluidic Droplet Coalescence,” by Ahn, et al., published as U.S. Patent Application Publication No. 2007/0195127 on Aug. 23, 2007, incorporated herein by reference in its entirety.

In one set of embodiments, a fluid may be injected into a droplet. The fluid may be microinjected into the droplet in some cases, e.g., using a microneedle or other such device. In other cases, the fluid may be injected directly into a droplet using a fluidic channel as the droplet comes into contact with the fluidic channel. Other techniques of fluid injection are disclosed in, e.g., International Patent Application No. PCT/US2010/040006, filed Jun. 25, 2010, entitled “Fluid Injection,” by Weitz, et al., published as WO 2010/151776 on Dec. 29, 2010; or International Patent Application No. PCT/US2009/006649, filed Dec. 18, 2009, entitled “Particle-Assisted Nucleic Acid Sequencing,” by Weitz, et al., published as WO 2010/080134 on Jul. 15, 2010, each incorporated herein by reference in its entirety.

U.S. Provisional Patent Application Ser. No. 62/961,097, entitled “Devices and Methods for Determining Nucleic Acids Using Digital Droplet PCR and Related Techniques,” by Weitz, et al., is incorporated herein by reference in its entirety.

In addition, the following documents are also each incorporated herein by reference in its entirety for all purposes: Int. Pat. Apl. Pub. No. WO 2016/168584, entitled “Barcoding System for Gene Sequencing and Other Applications,” by Weitz et al.; Int. Pat. Apl. Pub. No. WO 2015/161223, entitled “Methods and Systems for Droplet Tagging and Amplification,” by Weitz, et al.; U.S. Pat. Apl. Ser. No. 61/980,541, entitled “Methods and Systems for Droplet Tagging and Amplification,” by Weitz, et al.; U.S. Pat. Apl. Ser. No. 61/981,123, entitled “Systems and Methods for Droplet Tagging,” by Bernstein, et al.; Int. Pat. Apl. Pub. No. WO 2004/091763, entitled “Formation and Control of Fluidic Species,” by Link et al.; Int. Pat. Apl. Pub. No. WO 2004/002627, entitled “Method and Apparatus for Fluid Dispersion,” by Stone et al.; Int. Pat. Apl. Pub. No. WO 2006/096571, entitled “Method and Apparatus for Forming Multiple Emulsions,” by Weitz et al.; Int. Pat. Apl. Pub. No. WO 2005/021151, entitled “Electronic Control of Fluidic Species,” by Link et al.; Int. Pat. Apl. Pub. No. WO 2011/056546, entitled “Droplet Creation Techniques,” by Weitz, et al.; Int. Pat. Apl. Pub. No. WO 2010/033200, entitled “Creation of Libraries of Droplets and Related Species,” by Weitz, et al.; U.S. Pat. Apl. Pub. No. 2012-0132288, entitled “Fluid Injection,” by Weitz, et al.; Int. Pat. Apl. Pub. No. WO 2008/109176, entitled “Assay And Other Reactions Involving Droplets,” by Agresti, et al.; and Int. Pat. Apl. Pub. No. WO 2010/151776, entitled “Fluid Injection,” by Weitz, et al.; and U.S. Pat. Apl. Ser. No. 62/072,944, entitled “Systems and Methods for Barcoding Nucleic Acids,” by Weitz, et al.

In addition, the following are incorporated herein by reference in their entireties: U.S. Pat. Apl. Ser. No. 61/981,123 filed Apr. 17, 2014; PCT Pat. Apl. Ser. No. PCT/US2015/026338, filed Apr. 17, 2015, entitled “Systems and Methods for Droplet Tagging”; U.S. Pat. Apl. Ser. No. 61/981,108 filed Apr. 17, 2014; U.S. Pat. Apl. Ser. No. 62/072,944, filed Oct. 30, 2014; PCT Pat. Apl. Ser. No. PCT/US2015/026443, filed on Apr. 17, 2015, entitled “Systems and Methods for Barcoding Nucleic Acids”; U.S. Pat. Apl. Ser. No. 62/106,981, entitled “Systems, Methods, and Kits for Amplifying or Cloning Within Droplets,” by Weitz, et al.; U.S. Pat. Apl. Pub. No. 2010-0136544, entitled “Assay and Other Reactions Involving Droplets,” by Agresti, et al.; U.S. Pat. Apl. Ser. No. 61/981,108, entitled “Methods and Systems for Droplet Tagging and Amplification,” by Weitz, et al.; Int. Pat. Apl. Pub. No. PCT/US2014/037962, filed May 14, 2014, entitled “Rapid Production of Droplets,” by Weitz, et al.; and U.S. Provisional Patent Application Ser. No. 62/133,140, filed Mar. 13, 2015, entitled “Determination of Cells Using Amplification,” by Weitz, et al.

The following are also each incorporated herein by reference in its entirety: U.S. Provisional Patent Application Ser. No. 63/016,950, filed Apr. 28, 2020, entitled “High Throughput and High Sensitivity Detection of Disease Biomarkers and Pathogens,” by Chang, et al. and U.S. Provisional Patent Application Ser. No. 63/106,255, filed Oct. 27, 2020, entitled “Systems and Methods for Determining Viruses or Other Pathogens,” by Weitz, et al.

The following examples are intended to illustrate certain embodiments of the present disclosure, but do not exemplify the full scope of the disclosure.

### Example 1

The purpose of this example is to demonstrate a device that is able to detect very low levels of a pathogen very rapidly and very inexpensively, e.g., without the need for trained personnel, personal protective equipment or highly specialized equipment. The initial target is SARS-CoV-2, and this device can be used to provide very widespread, rapid and inexpensive testing with the same sensitivity as the current “gold-standard” test using quantitative polymerase chain reaction (qPCR).

The device in this example can also be used for any other nucleic acid test, including other viruses, bacteria, fungi or other nucleic acid targets. This includes flu, Ebola, any SARS species, MERS, or any other viral or bacterial species. One can change the primers for the nucleic acid amplification and adapt the initial loading of the sample into the device.

The device uses a self-contained microfluidic chip to which the sample is loaded. The sample may originate, for example, through saliva or through a nose swab. All the other reagents are contained on the chip. The detection is achieved in the device through an amplification step followed by a detection step. The amplification is through loop mediated isothermal amplification (LAMP), using fluorescent detection. The initial sample is compartmentalized into roughly 1 million drops or other compartments using droplet LAMP (dLAMP). The drops or compartments are arranged in an array on the chip and are then heated on a hot plate to 65° C. The fluorescent signal from the drops is detected with a small box that includes a sample holder to plug the chip into, an LED to excite the fluorescence, a dichroic to block the excitation light and a camera to image the array of drops and detect the fluorescent signal from the positive drops. The viral load is determined by counting the number of positive drops.

The goal of this compartmentalization is to increase the concentration of the target molecules, which may result in a more rapid test. The time taken to measure a detectable signal in this test is equivalent to the time taken to detect a measurable signal for a concentration 1 M times higher for a bulk sample. The estimated time is 5-8 minutes in this device. This represents a significant reduction in time.

This device provides a widely deployable, rapid screen that can be applied to everyone. For example, the test can be used to screen everyone entering a sports event, a theater or concert, an office building, an airport, or even a restaurant. If the results are coupled with a smartphone-based app, the test can be performed on a rolling time sequence, for example once per day, and if someone is negative, they would not need to repeat the test for the prescribed time.

RT-LAMP is an isothermal, rapid technique for the exponential amplification of RNA that avoids the thermal cycling and high temperatures required by RT-qPCR. RT-LAMP uses a set of four to six unique primers that, in the presence of DNA polymerase, repeatedly produce copies of target cDNA through the formation of loop structures. Replacing PCR with LAMP amplification represents a significant step forward for molecular nucleic acid testing. At the clinical laboratory test scale, the RT-qPCR test is considered a complex test where results can only be obtained by trained personnel. Current processing using the CDC approved diagnostic panel takes at least 3 hours, and usually much longer, to complete, including sample acquisition, RNA extraction, reagent preparation, and analysis of the viral N1, N2 and human RNase P genes. LAMP can amplify a single copy of RNA to 10⁹ in less than an hour. The amplification is much faster, even when there are large amounts of non-target RNA present, and even with “dirty” samples such as blood, serum, feces and nasal swabs. There is no need for equipment except a water bath or heat block at a temperature of 60-65° C., which avoids the thermal cycling and high temperatures required by the gold standard RT-qPCR which takes two hours to run. By simply replacing PCR with LAMP chemistry, significant gains in turnaround time can be achieved.

The microfluidic point-of-care device used in this example is based upon droplet LAMP (dLAMP) in which the sample is partitioned into drops. In dLAMP, single nucleic acids can be detected within picoliter-sized drops. The microfluidic dLAMP device has the advantage in massive high throughput testing by using simple, cheap, disposable devices. Under isothermal conditions, the amplification signal arising from a single virion can be rapidly collected and concentrated in a tiny drop. A signal is detected as soon as the response of individual drops is above background noise. This shortens the time necessary to see a positive signal compared to bulk. The method has higher high sensitivity compared to bulk allowing for a lower number of false negative results.

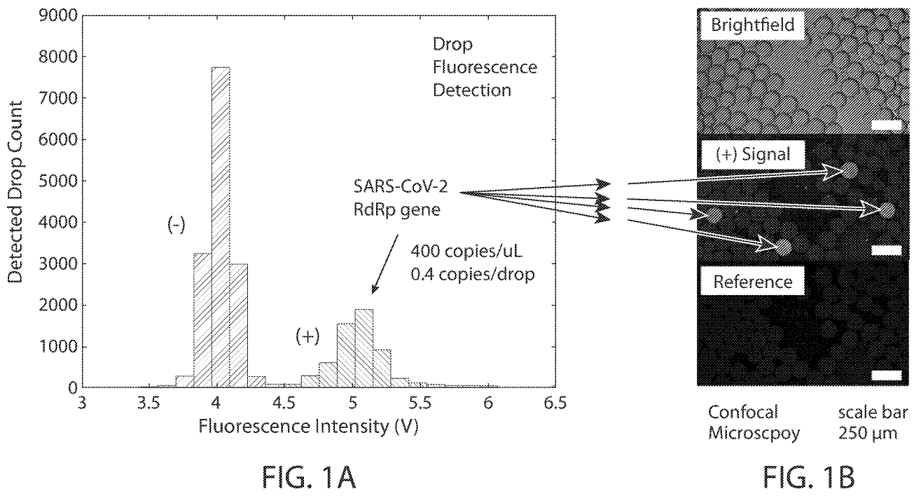

Preliminary data demonstrating dLAMP amplification in drops is as follows. dLAMP amplification of the SARS-CoV-2 gene can occur in drops (FIGS. 1A-1B). Here, the RdRp gene of SARS-CoV-2 was amplified at a concentration within the range of typical SARS-CoV-2 positive patient saliva samples (10¹ to 10⁶ copies/microliters). A 25 microliter solution of LAMP mixture containing a concentration of 400 copies/microliters or 0.4 copies/drop was partitioned into 120 micrometer-diameter drops. The drops were heated for 30 minutes at 65° C. and fluorescence amplification was measured using a qPCR machine. Amplification began in 15 minutes while holding the drops at constant temperature of 65° C. A custom-built microfluidic device was used. Optical detection was used to measure fluorescence of thousands of drops to quantify the number of positive drops containing a single RNA template and the number of negative drops containing no RNA copies. These numbers matched with expected Poisson statistics.

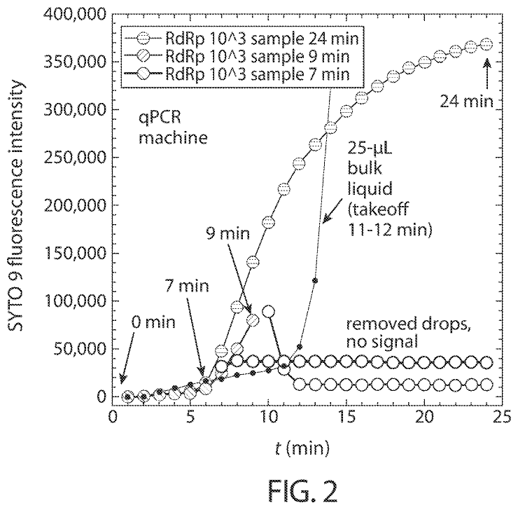

The microfluidic chip can be engineered to optimize drop size, number of drops, and provide high signal-to-noise. By partitioning the typical 25 microliter LAMP reaction into picoliter-sized drops, analysis time will be greatly reduced. Because of the small size of the drops, less time was needed to reach a detectable concentration of nucleic acid from a single template molecule. Results showed that when a solution of 10³ copies/mL of RdRp gene is partitioned into ˜380,000 of 50 micrometer droplets, amplification take-off in drops occurred between 7-9 minutes in drops compared to 11-12 minutes in bulk (FIG. 2).

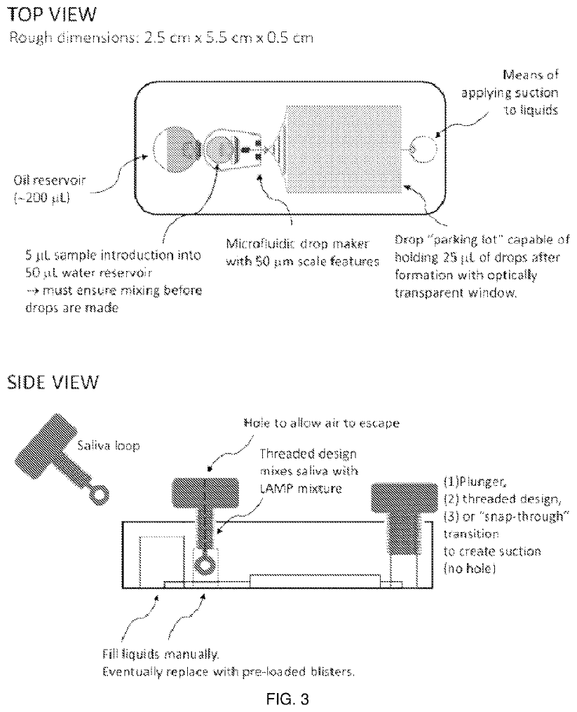

The device in this example used a saliva or nasal mucus sampling loop or swab and a small microfluidic chip. The microfluidic chip included an orifice for loop or swab insertion, a chamber for mixing the sample with an aqueous solvent containing nucleic acid amplification reagents, a system for partitioning the aqueous liquid containing the sample into individual microscale volumes, and a system for creating a pressure difference to drive oil and aqueous solvents through the device to partition the aqueous solvent into individual microscale volumes. One possible representation of the sampling loop or swab and chip is shown in FIG. 3.

The device can be operated by inserting the sampling loop or swab into the chip and driving flow by means of a plunger or other mechanical actuator. The mechanical actuator will act by generating a positive or negative pressure. Nasal mucus or saliva sample volumes on the order of 1 microliter to 10 microliters can be mixed with appropriate nucleic acid amplification reagents on the order of tens of microliters, formed into monodisperse drops with diameters of less than 100 micrometers, and collected downstream within a cavity in the chip. Fluorinated or silicone oil volumes on the order of hundreds of microliters act as the continuous phase, separating drops. Once formed, the drops can be heated to a well-defined temperature (e.g. 65° C.) for a well-defined time period, e.g., on the order of minutes, to amplify nucleic acids encapsulated in each drop. Amplification will result in binding and activation of a fluorescent molecule, which will be assayed by illumination with visible light and collection of a fluorescent signal. Drop creation, heating, and fluorescence detection can be performed by insertion of the chip in a separate box capable of mechanical actuation, heating, and with the necessary optical components for fluorescence imaging. Optical components may include a light source, filters, diffusers, and a camera. The imaging device may also include wireless communication network capabilities. Alternatively, the drop creation will be done by the user without any accessories other than those on the chip itself, the heating will be done on a separate hot plate, and only the detection and any network connectivity will be done by the separate box.

The device may be mass produced, for example, using injection molding, die cutting, or other fabrication methods and adhered together using laser welding or pressure sensitive adhesive. The device may be composed of mechanically stable, chemically resistant, optically transparent materials like cyclic olefin copolymer, which is widely used for point-of-care diagnostic devices. Other materials may also be used. The chip may include liquid filled blister packs or other liquid reservoirs. The chip may also contain resistive heating elements or other components for heating the contents of the chip.

FIGS. 1A-1B show a digital RT-LAMP detection and quantification of SARS-CoV-2 RdRp gene RNA standard. FIG. 1A shows drop fluorescence detection of a total of ˜21,000 drops of which 6,600 are positive (containing 1 or more RNA copies) and 14,400 are negative (containing no RNA copies). Poisson statistics estimates 400 copies/mL (0.4 copies/drop) in agreement with the known RNA sample concentration determined independently using bulk RNA RT-LAMP amplification standard curve fitting methods (FIGS. 1A-1B). Results corresponded to a primary sample concentration of 10⁴ copies/mL, well within the range of typical SARS-CoV-2 positive patient saliva samples (10¹ to 10⁶ copies/mL). FIG. 1B shows a brightfield, (+) signal, and reference (ROX dye) confocal micrographs of 120 mm drops used in digital RT-LAMP analysis. Bright green drops are positive for the presence of at least 1 copy of RdRp RNA standard and dim green drops contain no RdRp copies.

FIG. 2 shows dLAMP detection and quantification of SARS-CoV-2 RdRp gene RNA standard. Amplification take-off in drops begins several minutes before bulk (between 7-9 minutes in drops versus 11-12 minutes in bulk). Four PCR tubes of 25 microliters, each of 50-micrometer drops, were created containing RT-LAMP primers and reagents and taken out of a qPCR machine that is paused at various time intervals (0, 7, 9, 24 minutes) for fluorescence imaging.

FIG. 3 illustrates one potential embodiment of the device.

### Example 2

The device in this example improves point-of-care diagnostics by providing rapid readout in 5 minutes on a cartridge. The simplicity, speed, and sensitivity makes the device in this example and the corresponding app useful for testing asymptomatic individuals, enabling quarantine, contact tracing, early treatment, decreasing the probability of secondary infections, etc.

The SARS-CoV-2 virus has rapidly evolved into a pandemic that is threatening public health, economics, and quality of life worldwide. To control the spread of the virus, population-level testing of millions of people a day is imperative to rapidly identify infected individuals in a broad surveillance effort to stop the chain of transmission, allow contact tracing, etc. Extensive, widely available, inexpensive and rapid tests are needed. Broad surveys of the population using at least millions and potentially tens of millions of tests per day will be necessary to identify resurgence of the virus and halt localized outbreaks. Currently, the “gold-standard method” for the detection of SARS-CoV-2 is quantitative reverse transcription polymerase chain reaction (qRT-PCR) pioneered by the US CDC. The qRT-PCR test requires significant reagent consumption, specialized equipment, trained operators, and several hours to perform. To expand surveillance across a global scale will call for new strategies for tests that are inexpensive, require minimal reagents, decrease assay time, and allow for simple point-of-care (POC) monitoring without need of trained personnel.

To this end, this example illustrates systems and methods for accelerating and bypassing traditional clinical-level diagnostics by using devices to massively test the population and the app to monitor the tests.

The device in this example is a microfluidic chip that is a self-contained, fast (<5 minutes) and scalable POC diagnostic test. The results are read and analyzed by a cellphone, allowing a connected analysis and reporting system through the app. The basis of the detection is isothermal PCR through the use of RT-LAMP, or reverse transcriptase loop-mediated isothermal amplification. LAMP is an isothermal, single tube technique for the exponential amplification of RNA. Operating at a temperature of 60-65° C., RT-LAMP removes the bottleneck of thermal cycling and high temperatures required by any regular PCR-based method. The sensitivity is achieved by performing digital droplet-based LAMP (ddLAMP) in which the sample is partitioned into drops; thus, a signal is detected as soon as the response of individual drops is above background noise. By using large numbers, the drops can be very small, reducing the time needed for a detectable response. The target for the time for the whole test is 5 minutes. In addition, by using large numbers of small drops, the test is very sensitive. To ensure ease of use, sampling will be performed using saliva, making sample collection very fast and very simple, allowing widespread compliance without the need of highly trained operators (although other sampling techniques or biological fluids can also be used). The device in this example is be self-contained, with the whole test carried out using only the chip. It is therefore highly scalable, allowing millions of tests to be carried out daily. A hot plate that maintains the temperature at the required value (65° C.) while the LAMP amplification is carried out may be used. In addition, a small read-out unit may be used, which ensures the chip is in a dark environment and which allows the use of an excitation LED to illuminate the chip and a dichroic to separate the excitation from the fluorescent readout. The readout box has a mounting for a cellphone which serves as both the detector and performs the analysis of the results.

This may allow, in some embodiments, for massive testing of the population, including those who do not have COVID-19. The device in this example employs a nucleic-acid based test which is suitable to test patients who are infectious. The goal of this example is to allow for testing of large numbers of people (e.g., millions) at least once every two weeks. This would demand performing approximately 25 M tests per day. However, this is reasonable because the test is self-contained on a chip and very simple to perform; thus, the testing can be highly decentralized and performed in many locations. The concept of this example is that locations where people gather would have restricted entry. This would include, for example, stores, restaurants, malls, stadiums, office buildings, etc.

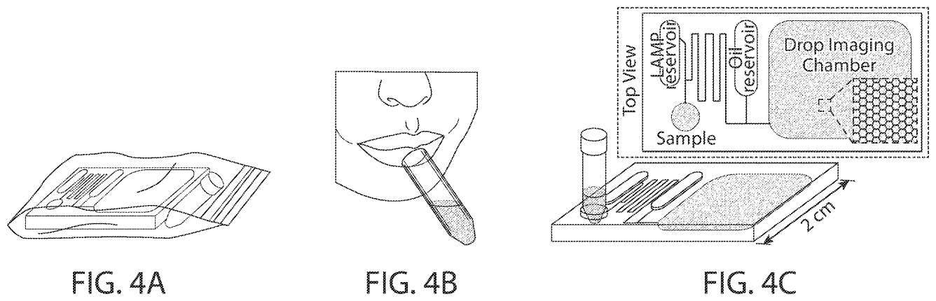

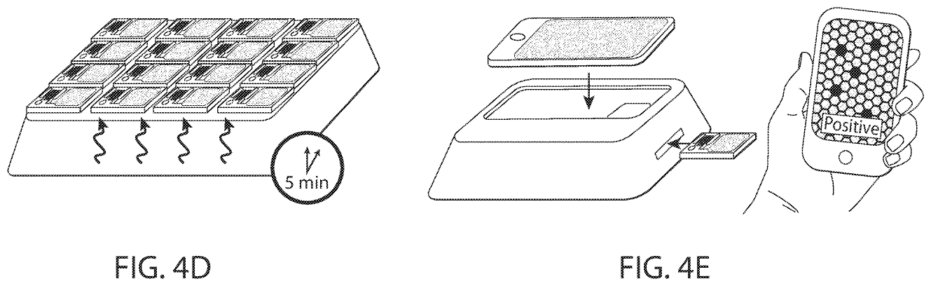

A schematic illustrating how the device in this example works is shown in FIG. 4. An app may be used, e.g., on a cellphone. A subject would spit into a self-contained tube on the chip. A small amount of the saliva would be added to the LAMP reagents, and an onboard fluid manipulation system would mix the reagents and sample and form the drops. The onboard fluid manipulation system would consist of a small air plunger, or a small squeeze pouch operated with two fingers. The chip would then be placed on the heating plate for 5 minutes, and then inserted in the readout box to allow all the drops to be imaged using fluorescence. The cellphone would collect the image and performs the analysis. A negative result would cause a ‘clear’ signal to be sent to the phone of the person who registered for the test. The next time the recipient went into a restricted location, the ‘clear’ signal would be displayed by their cellphone, allowing entry without a further test. The device in this example, and the essential equipment could be scaled to allow millions to be deployed and testing to be performed on a large fraction of the population each day.

FIGS. 4A-4E illustrate the device of one embodiment. FIG. 4A shows the device of one embodiment and spit attachment wrapped in sterile packaging. FIG. 4B illustrates a person expressing saliva through a spit attachment, filling a sample reservoir that is combined with preloaded assay reagents on the device of one embodiment. FIG. 4C shows millions of drops occupying positions on sample array. FIG. 4D illustrates the loaded device placed on a 65° C. hotplate for around 5 minutes for the reaction to reach completion in each drop. FIG. 4E shows the device of one embodiment inserted into the read-out box which illuminates the drop array. A photograph is uploaded using a smartphone app to analyze result and give a ‘clear’ or ‘not clear’ signal.

### Example 4

The goal of this example is to demonstrate a test that is cheap, simple, and fast enough to be used in a distributed fashion. For example, by screening everyone who enters a location where people congregate and the risk of infection is high, it ensures that these locations are safe and free of transmission of virus. Moreover, when someone does have the virus, identification will be early enough to more easily treat the infection, to isolate, and to perform contact tracing to limit spread through selective quarantines.

Currently, there are >100 molecular tests for SARS-CoV-2, but these methods are unsuitable for broad, routine population-wide surveillance. This is a need met only by the most rapid, inexpensive, point-of-care (POC) tests. Serological test kits are cheap, disposable, and can routinely produce results in <15 min. However, serology testing cannot identify recently infected and infectious yet asymptomatic persons, which can only be performed by nucleic acid testing of the virus. Over 90% of rapid POC molecular diagnostics for SARS-CoV-2 rely upon the RT-PCR reaction, which despite being a gold standard assay in the lab, is a much slower and more costly method than alternatives such as isothermal amplification. Global supplies of RT-PCR components are highly limited, so a test for millions must significantly reduce reagent consumption per assay. Reaction chemistry in RT-PCR is slow, requiring 45 minutes to complete.

Compared to other tests, the devices in this example feature a low limit of detection (LOD) down to a single virus in a picoliter-sized drop based on prior data. Test results are available in <5 minutes. The devices in this example have the advantage in massive high-throughput testing by using simple, cheap, disposable devices that can be read out hundreds to thousands at a time with the use of much smaller total reagent volumes. 1/10 of the volumes offered by current state-of-the-art POC tests is estimated. The devices in this example are poised to disrupt the diagnostic marketplace by offering sensitivity down to the lowest titers while avoiding false negatives encountered by qRT-PCR. The device in this example can detect very low viral titers to rapidly identify asymptomatic or newly infected individuals. For example, it would be deployable at already-existing security checkpoints common throughout the world.

Isothermal RT-LAMP is faster compared to qRT-PCR. RT-LAMP is an isothermal, rapid technique for the exponential amplification of RNA that avoids the thermal cycling and high temperatures required by qRT-PCR. RT-LAMP uses a set of four to six unique primers that in the presence of DNA polymerase repeatedly produce copies of target cDNA through the formation of loop structures. Replacing PCR with LAMP amplification represents a significant step forward for molecular nucleic acid testing. At the clinical laboratory test scale, the qRT-PCR test is considered a complex test where results can only be obtained by trained personnel. Current processing using the CDC approved diagnostic panel can take up to 4 hours to complete, including sample acquisition, RNA extraction, reagent preparation, and analysis of the viral N1, N2 and human RNase P genes. This approach is extremely limited to approximately 100 tests per day at a local hospital using two 96-well qRT-PCR machine, which requires three RNA extractions a day and three trained technicians. By contrast, LAMP can amplify a single copy of RNA to 10⁹ in less than one hour. Results show that amplification of SARS-CoV-2 genes can begin in 5 minutes. The amplification is much faster, even when there are large amounts of non-target RNA present, and even with “dirty” samples such as blood, serum, feces, and nasal swabs. In addition, RT-LAMP is 100-fold more sensitive than traditional PCR. There is no need for equipment except a water bath or heat block. Placed on, e.g., a hot plate at a temperature of 60-65° C., the device in this example avoids the thermal cycling and high temperatures required by the gold standard qRT-PCR which takes two hours to run and can have a false negative rate of up to 30%. By simply replacing PCR with LAMP chemistry, significant gains in turnaround time can be achieved.

Drops (ddLAMP) may reduce reaction time, in some embodiments. An RT-LAMP reaction takes approximately 20 minutes to complete, already a substantial decrease in time compared to qRT-PCR. The device in this example reduces this time using a digital droplet microfluidic array. This is a rapid, highly sensitive method for the detection of virus using digital droplet LAMP (ddLAMP). By partitioning the typical 25 mL LAMP reaction into a million drops, analysis time is reduced. Because of the small size of the drops, less time is needed to reach a detectable concentration of nucleic acid from a single template molecule. Based on results, the nucleic acid content is increased 10-fold every 135 seconds. Therefore, it is estimated that reducing the reaction volume by 10⁶-fold reduces a 20 minute assay time by about 13-14 minutes. In addition, in some cases, the assay can confirm both positive and negative samples within 5 minutes. This short assay time can enable unprecedented rapid and scalable screening.

The device in this example may be sensitive to a single virus in a drop, under some conditions. The device in this example can generate millions of picoliter droplets containing a mixture of patient samples and LAMP reagents which are loaded into a predefined well array. It is well known that ddPCR is much more sensitive compared to qPCR and that ddLAMP is much more sensitive compared to LAMP. The device in this example employs a fluorescence assay for higher sensitivity and faster acquisition of data with very high signal-to-noise. The amplification signal is collected and concentrated in a tiny drop. This shortens the time necessary to see a positive signal. One bright drop in a million is all that is necessary to give a positive result. Thus, the high sensitivity enables detection of virus from asymptomatically contagious people who have no obvious illness indicators, yet are shedding virus. One bright drop will yield either a ‘clear’ or ‘not clear’ signal.

RNA extraction is not required. Results indicate that an RNA chemical extraction step may be avoided in some cases using RT-LAMP. There are also results that RNA extraction is not needed to detect a single influenza virus in a drop prior to performing digital droplet PCR (ddPCR). The RNA within an enveloped virus such as influenza or SARS-CoV-2 can be accessed through heat lysis.

Drops use very little LAMP reagent. The typical SARS-CoV-2 viral load in positive patient saliva samples is 10¹ to 10⁶ copies/mL. To design an assay which can reliably detect the lowest viral load of ˜10 virions/mL, a sample 1 mL of saliva containing about −10 virions is needed. The saliva sample is diluted into as little as 10 mL of RT-LAMP reagents, about 2.5× less than conventional RT-LAMP assays, before partitioning the mix into 10⁵ drops of 10 pL (about 27 mm in diameter) in this example device. This partitioning increases the effective concentration by about 10⁴ fold, from 10 virions/mL to about 10⁵ virions/mL, inside drops that contain a viral particle. This workflow decreases the utilization of scarce reagents such as enzymes and dNTPs multiple-fold while reducing the time from sampling to detection.

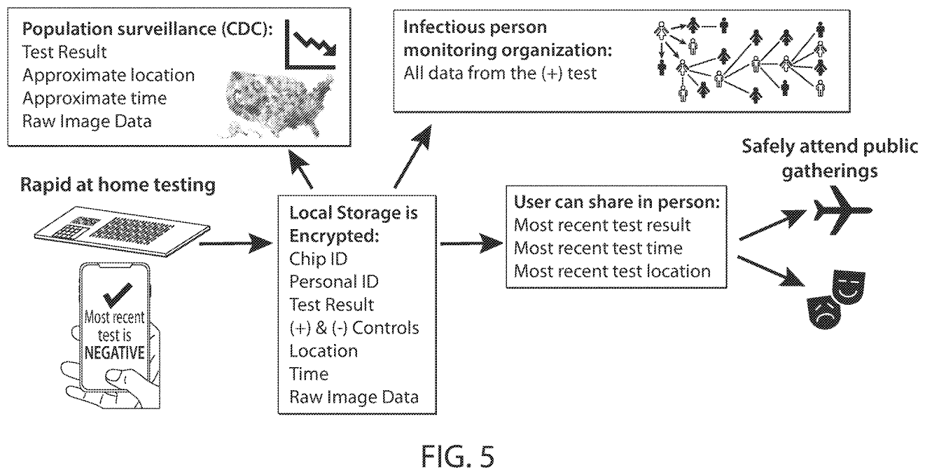

The device in this example may be designed in a way such that the test results are readable with a smartphone, in some embodiments. As a non-limiting example, the disposable chip may have a unique QR code which a user can scan with their phone, linking the specific chip to the user. The data acquisition and storage may be done, for example, with a standalone app. A schematic showing how an example app works is shown in FIG. 5 (although other app designs can also be used in other embodiments). One goal, for example, may be to protect the user's privacy while also preserving public health and safety. To ensure this privacy, data can be locally stored and locally encrypted. For each test, the app can store a QR code that identifies, for example, data such as the device, the result of positive/negative internal controls, the result of the test itself, the location and time of the test, a personal identifier, a photo of the droplet array (raw data), or other pertinent data.

FIG. 5 shows that the app securely stores results of test data for infectious person monitoring.

The ability to amplify for the SARS-CoV-2 virus can be demonstrated in drops. In this example, the device is used for detection of SARS-CoV-2 from patient samples. The device in this example is optimized for drop size, reagent mixtures, and chip design. Data demonstrates the RdRp and E gene of in vitro transcribed SARS-CoV-2 can be detected in 100 mm diameter drops using LAMP. Amplification starts as early as 5 minutes and takes no more than 20 minutes in bulk. The assay is optimized to detect a positive result with the SARS-CoV-2 virus within 5 minutes in drops. The SARS-CoV-2 virus has been cultivated, produced, and quantified for infectivity in BSL-3 facilities. Preliminary results demonstrate a LOD of 0.4 copy/mL in bulk. The LOD in drops is determined using spiked concentrations and is determined in the expected background of the virus to test inhibitory effects. Although the goal is to test saliva, the possibility of swab sampling is also possible, as both saliva and swab are the most compatible methods for self-testing. Only a single positive drop needs to be imaged, which is bright and easily detectable. Various drop-making schemes are possible, including microfluidic chips that can rapidly create drops in a parallelized manner. Various materials are possible for chip design, including glass, hot embossed or injection molded materials that can support heating, e.g., at a temperature of 60-65° C. for approximately 10-20 minutes. A box is created containing a hot plate, dichroic filter, excitation light source, and downstream cell phone detection.

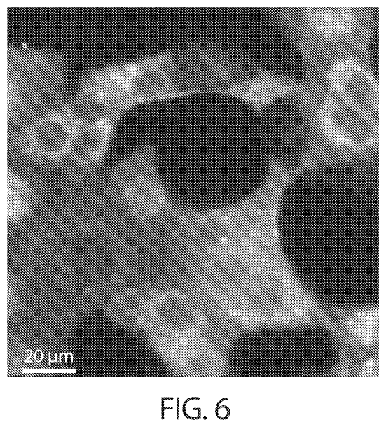

SARS-CoV-2 viral infection in Vero E6 cells has been successfully visualized (FIG. 6) and fundamental research on coronaviruses has been performed.

FIG. 6 illustrates a micrograph of Vero host cells infected with SARS-CoV-2. Infected Vero host cells are stained for identification of SARS-CoV-2 nucleoprotein N and spike protein S at 24 hours post infection (hpi). DNA localized within cell nuclei can also be seen.

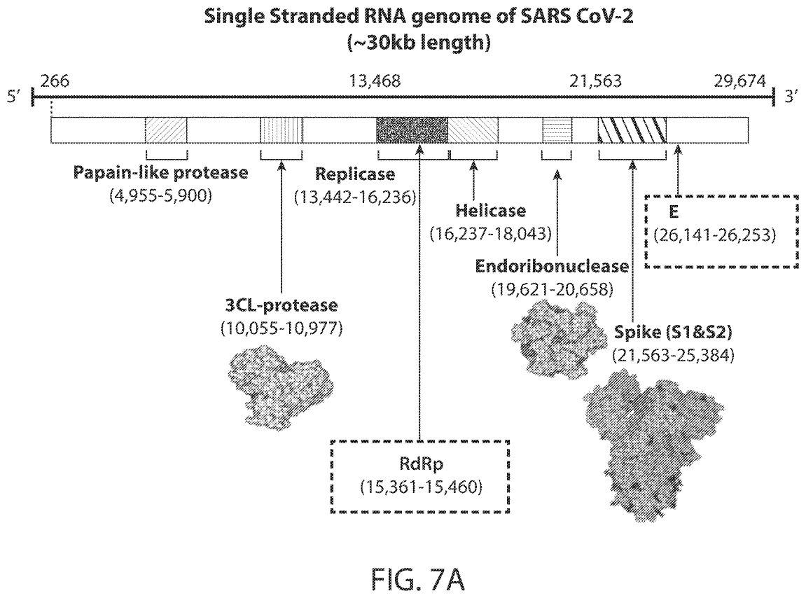

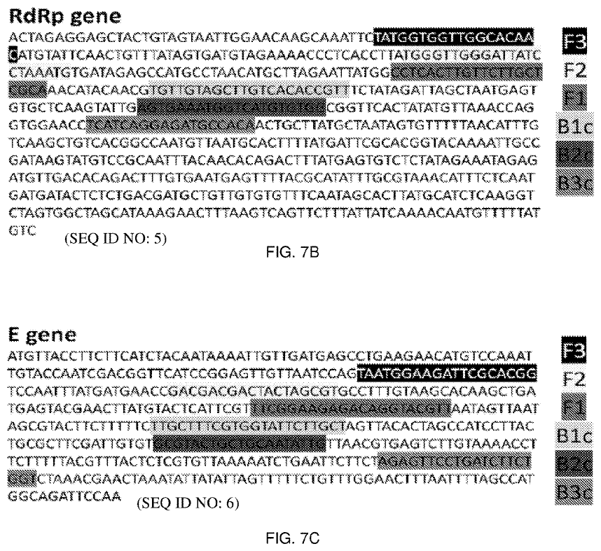

FIGS. 7A-7C show RT-LAMP primer sets for detection of SARS-CoV-2 E and RdRp gene sequences. FIG. 7A is a schematic of full ˜30 kb single stranded RNA genome of SARS-CoV-2. Target locations of RdRp and E gene sequences are highlighted in dashed boxes. FIGS. 7B and 7C show 5′ to 3′ sequences of SARS-CoV-2 RdRp and E genes revealing LAMP primer binding sites F3, F2, F1, B1c, B2c, and B3c. The sequences in FIGS. 7B and 7C correspond to SEQ ID NO: 5 and SEQ ID NO: 6, respectively.

Standard qRT-PCR amplification of SARS-CoV-2 in bulk and drops. qRT-PCR detection of influenza virus in drops has been performed. Standard qRT-PCR has been performed upon in vitro on transcribed E and RdRp genes of SARS-CoV-2 as well as the full-length genomic SARS-CoV-2 RNA. Amplification of SARS-CoV-2 RdRp RNA and full-length genomic RNA in drops has been demonstrated using qRT-PCR (data not shown).

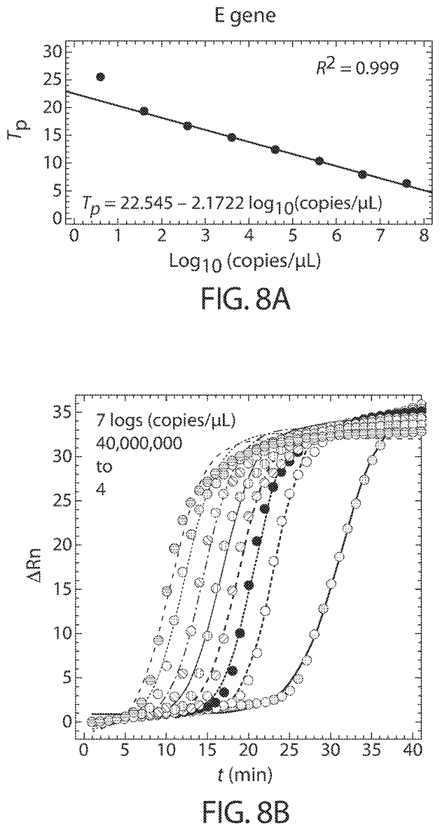

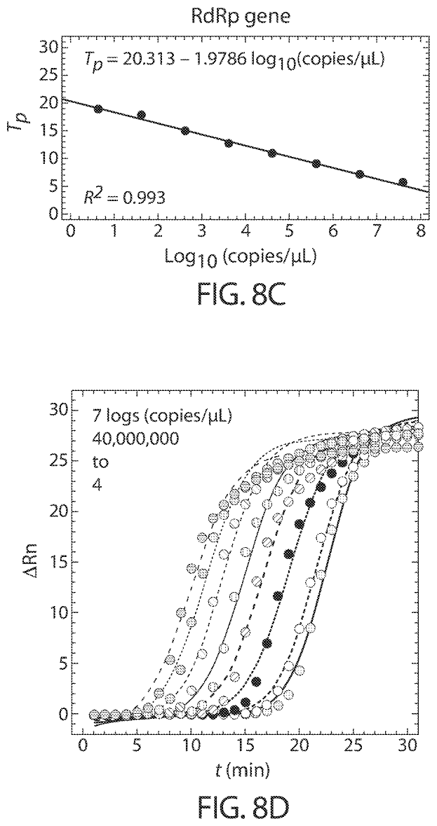

RT-LAMP amplification of RdRp and E genes of SARS-CoV-2 in bulk. Next the virus was amplified in drops using the rapid isothermal RT-LAMP method. RT-LAMP primer sets were designed for the SARS-CoV-2 E and RdRp genes (FIGS. 7A-7C) which correspond to the in vitro transcribed genes used in qRT-PCR and were tested using the NEB WarmStart LAMP kit on dilutions of each gene. From the dilution series, amplification curves were obtained as a function of time over 7 logs of RNA concentrations, from 4×10⁻¹ up to 4×10⁷ copies/mL (FIGS. 8A-8D). Both linear fits of the standard curves are excellent with R²>0.99 between 7 logs of standard RNA concentrations. The standard curves can be used to determine concentrations of unknown SARS-CoV-2 patient samples. The experiments demonstrate excellent efficiencies and data shows sensitivities down to 0.4 copies/mL in one trial. For both the E gene and the RdRp gene, amplification occurs as early as 5 minutes at 10⁷ copies/mL.

FIGS. 8A-8D illustrate standard curves generated using RT-LAMP primer sets and SARS-CoV-2 in vitro transcribed RNA standards (FIG. 8A) and (FIG. 8C). Standard curves calibrated using (FIG. 8A) E gene and (FIG. 8C) RdRp gene. Both linear fits are excellent with R2>0.99 between 7 logs of RNA standard concentrations. Inset fitting curve functions can be used for determining concentrations of unknown SARS-CoV-2 patient samples. (FIG. 8B) and (FIG. 8D) RT-LAMP amplification curves measured in qPCR machine over 7 logs of RNA standard concentrations 4×10⁷, 10⁶, 10⁵, 10⁴, 10³, 10², 10¹, and 10⁰ copies/microliters (going left to right) for (FIG. 8B) E gene and (FIG. 8D) RdRp gene. Fits are of the form ΔRn (t)=a+((k−a))/((1+e\hat{ }^{(-b(t-m}))) where parameter m and b can be used to calculate Tp from T_{P}=m−2/b and thereby generate standard curves (FIG. 8A) and (FIG. 8C).

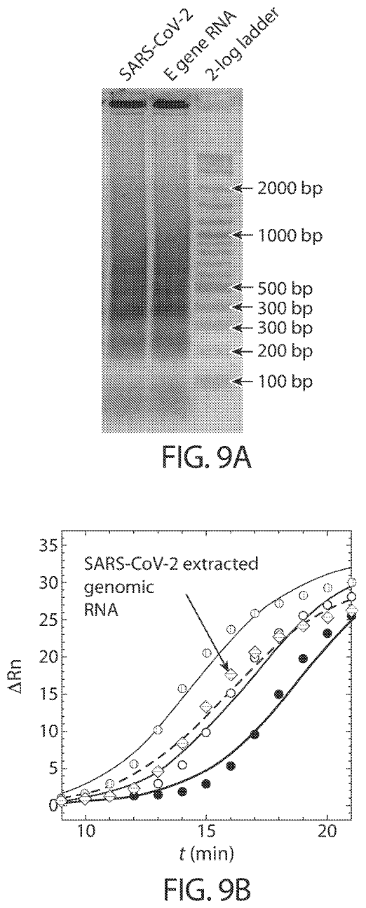

RT-LAMP amplification of the E gene from full length SARS-CoV-2 genomic RNA. It is demonstrated that the E gene is amplified from the full-length purified genome from SARS-CoV-2 virus (FIGS. 9A-9B). This concentration is approximately 1.4×10⁵ copies/mL.

FIGS. 9A-9B show that SARS-CoV-2 genomic RNA is detected using RT-LAMP E gene primer set. FIG. 9A illustrates the RT-LAMP amplified product of SARS-CoV-2, in vitro transcribed E gene RNA standard, and 2-log ladder separated on 3.0% wt agarose gel in 1×TAE buffer. Amplicon bands of SARS-CoV-2 and E gene RNA standard are in excellent agreement and indicate precise amplification with no detectable side products. FIG. 9B shows SARS-CoV-2 genomic RNA amplifying starting at 10 minutes. Fit to E gene standard curve (FIG. 9A)) estimates 140,000 copies/mL test concentration and 3,500,000 copies/mL primary sample concentration. SARS-CoV-2 genomic RNA sample (diamonds, pink dashed line fit) shown along with 10⁶ (left circles/line fit), 10⁵ (middle circles/line fit), and 10⁴ (right circles/line fit) E gene in vitro transcribed RNA standards.

Data demonstrates LAMP amplification in drops. Results demonstrate that LAMP amplification of the SARS-CoV-2 gene can occur in drops. Here, the NEB WarmStart LAMP kit was used to amplify for the RdRp gene standard at a concentration within the range of typical SARS-CoV-2 positive patient saliva samples (10¹ to 10⁶ copies/mL). A 25 microliter solution of LAMP mixture containing a concentration of 400 copies/mL or 0.4 copies/drop was partitioned into 120 micrometer diameter drops. The drops were heated in a qRT-PCR machine for 30 minutes and fluorescence amplification was measured. Amplification began in 15 minutes while holding the drops at constant temperature of 65° C. A microfluidic fluorescence drop reader was used to quantify the number of positive drops containing a single RNA template and the number of negative drops containing no RNA copies and these numbers matched with expected Poisson statistics. Here, 120 micrometer diameter drops were used to partition the sample. Smaller drops should allow for faster detection of signal due to the rapid accumulation of amplification produced in a lower effective volume.

The assay is verified using spiked in SARS-CoV-2 virus. The assay is tested using positive patient samples stored in the BSL-3 laboratory. Direct saliva to assay is also tested for inhibitory effects. IRB and IBC protocols are in place for both swab and saliva research and collection of patient samples.

Three steps are demonstrated: the saliva sample are mixed with LAMP reagents, drops are produced, and a drop array is created to hold the drops while they are incubated to then read the results. Drop makers can be created that are scaled in number to allow creation of 1 million drops in short times of less than 30 seconds. Drop makers have also been developed that can be successfully operated by handheld syringes. Designs for the initial sample handling are incorporated directly into the drop maker portion of the chip. The use of saliva simplifies the design of this section of the device in this example. Finally, some droplet arrays are adapted to the needs of the chip. All of the components are tested using soft lithography, but can be designed assuming that the production chip is injection molded or made in some similar, scalable fashion. The use of drops to compartmentalize the sample has the advantage of improving the signal to noise, and of decreasing the time of the assay to provide a result. The target goal for the device in this example is to optimize this to reduce the time to 5 minutes.

### Example 4

This example shows, a range of additives were investigated for optimizing droplet quantitative reverse transcriptase PCR (qRT-PCR) and allowing quantification of influenza A virus (JAY) in 100 micrometer diameter drops. Additive combinations of PEG, Tween-20, BSA, and betaine were screened for their ability to stabilize drops from coalescence and limit diffusion between neighboring drops. Together with 3 wt % (w/w) of PEG-PFPE2 surfactant in the oil phase, an additive combination of 1% w/v Tween-20, 0.8 μg/μl BSA, and 1 M betaine in a standard qRT-PCR mixture was found to be optimal for stabilizing drops and limiting dye diffusion under qPCR thermocycling between 60° C. and 95° C. Using Tween-20/BSA/betaine as additives, five orders of magnitude of in vitro transcribed viral RNA was resolved between drops ranging from 10⁻¹ to 10⁴ copies per drop (cpd). Drops containing low (10¹ cpd) and high (10⁴ cpd) viral RNA concentrations were thermocycled using a standard qPCR machine and the qRT-PCR amplification curves were comparable to constructed curves obtained from drops sampled at various cycles using epifluorescence microscopy. The optimized droplet qRT-PCR assay was used to quantify the number of A/California/07/2009 (H1N1) JAY genomes present in the supernatant from infected A549 cells. At 24 hours post infection (hpi), six dilutions of supernatant were prepared from infected cells to determine the detection limit for live virus. Using ddPCR analysis, JAY RNA was quantified directly from infected cells across four orders of magnitude. It was found that the limit of detection was at a dilution that corresponds to 0.320 cpd, demonstrating that the ability to detect down to a single viral genome in a drop. In addition, viral RNA was amplified from supernatant collected directed from infected cells without an RNA extraction step. This direct PCR method will have impact in reducing the number of steps for future work performing droplet qPCR of viruses.

Materials and Methods. TaqMan qRT-PCR Reactions. The sequences of qRT-PCR amplification primers for the JAY Matrix gene (M gene) were as follows: Matrix gene forward primer 5′-GACCRATCCTGTCACCTCTGAC-3′ (SEQ ID NO: 1), Matrix gene reverse primer 5′-AGGGCATTCTGGACAAATCGTCTA-3′ (SEQ ID NO: 2). The sequence of Matrix gene TaqMan probe was 5′-/FAM/TGCAGTCCTCGCTCACTGGGCACG/BHQ1/-3′ (SEQ ID NO: 3). Primers and probe were ordered from Eurofins Operon and were prepared as 100 micromolar stocks. The working stocks of the primers were 25 micromolar with a final concentration of 400 nM. The working stock of the probe was 10 micromolar with a final concentration of 200 nM. Samples were amplified using a SuperScript III Platinum One-Step qRT-PCR kit (Invitrogen 11732-020) with a final reaction volume of 25 microliters. Each reaction mix contained 0.05 micromolar ROX reference dye, 2 mM MgSO4 and 0.32 U/microliter SUPERase RNase Inhibitor (Invitrogen AM2694), and 2.5 microliter of RNA or supernatant. Tested additives were added to the qRT-PCR reaction mix at the following concentrations: 1% w/v Tween-20 (Calbiochem 655204-100 mL), 0.8 microgram/microliter BSA (Fisher BP675-1), 2.5% w/v PEG-6K (Acros Organics 192280010), and 1 M betaine (Sigma B0300-1VL). Thermocycling was performed in a qRT-PCR machine (Quantstudio 3, Applied Biosystems) with the following cycling conditions: 1 cycle for 30 minutes at 60° C., 1 cycle for 2 minutes at 95° C., and 40 cycles between 15 seconds at 95° C. and 1 minute at 60° C.

In vitro Transcribed RNA. To quantify the amount of viral RNA in each drop, standard curves were generated for each run using serial dilutions of in vitro transcribed IAV segment 7 (Matrix gene). The nucleic acid copy number in each reaction or drop was extrapolated from the standard curve. To generate the in vitro transcribed RNA, a gBlock containing a T7 promoter (underlined), forward and reverse primer sites (italicized), and probe sequence (bold) for the Matrix gene was ordered from IDT:

(SEQ ID NO: 4)
5′-GTCTAATACGACTCACTATAG GACCAATCCTGTCACCTCTGAC
TGCAGTCCTCGCTCACTGGGCACG
TGCTTCATCGCGAACTGCTTCGCGGATGCCATCGTCATGGCCACGAGGA
TATGTAAG
AGT TAGACGATTTGTCCAGAATGCCCT-3′.

The Matrix gene was transcribed in vitro using a MEGAscript™ T7 RNA Synthesis Kit (Ambion, AM1333) and purified over a GE Illustra Sephadex G-50 NICK column. The RNA was then DNase treated and precipitated with ammonium acetate/ethanol according to the manufacturer's instructions. RNA concentration was quantified using a NanoDrop spectrophotometer and used to determine the copy number per microliter.

Microfluidic device fabrication. Microfluidic devices for making 50 micrometer and 100 micrometer diameter drops were fabricated by patterning SU-8 photoresist (Microchem SU-8 3050) on silicon wafers (University Wafer, ID #447, test grade) using standard photolithography techniques. Polydimethylsiloxane (PDMS) (Sylgard 184) at 10:1 mass ratio of polymer to cross-linking agent is poured onto the patterned device master molds. Air was purged from the uncured PDMS by placing the filled mold in a vacuum chamber for at least 1 hour. The PDMS was cured in an oven at 55° C. for 24 hours and then removed from the mold with a scalpel. Ports were punched into the PDMS slab with a 0.75 mm diameter biopsy punch (EMS Rapid-Core, Electron Microscopy Sciences). The PDMS slab was bonded to a 2-in by 3-in glass slide (VWR micro slides, cat. #48382-179) after plasma treatment (Harrick Plasma PDC-001) for 60 seconds at high power (30 W) and 700 mTorr oxygen pressure. The drop making devices were made hydrophobic by flowing Aquapel (Pittsburgh Glass Works) through the channels, followed by blowing the channels with air filtered through a GVS ABLUO™ 25 mm 0.2 μm filter (Fisher Scientific) before baking the devices in an oven at 55° C. for 1 hour.

Drop encapsulation. Flow-focusing drop making devices were used to make 50 micrometer diameter drops for the drop stability and PCR dilution experiments and 100 micrometer diameter drops for infected supernatant experiments. The dispersed phase had qRT-PCR mix containing a range of concentrations (10³ to 10⁸ copies/microliters) of in vitro transcribed M gene RNA and different combinations of additives (no additives, Tween-20 only, BSA only, PEG-6K only, betaine only, Tween-20/BSA, Tween-20/PEG-6K and Tween20/BSA/betaine with concentrations as previously described). The continuous phase consisted of a 3 wt % (w/w) solution of PEG-PFPE2-based surfactant (RAN Biotechnologies, 008-FluoroSurfactant) in fluorinated HFE7500 oil (3 M). The dispersed and continuous phases were loaded in 1-mL syringes and injected into the drop making microfluidic devices at a flow rate of 800 microliters/h and 1600 microliters/h, respectively, using syringe pumps (New Era NE-1000) controlled by a custom LabVIEW (2015) program to generate drops. A total volume of 60 microliters composed of approximately 20 microliters of drops and 40 microliters of oil were collected in 100 microliters PCR tubes (Applied Biosystems). After the drops were collected, the PCR tubes were promptly placed in the qRT-PCR machine (Applied Biosystems QS3) and thermocycled.

Endpoint brightfield and fluorescence imaging of drops containing PCR additives combinations. Thermocycled drops were imaged by injecting 20 microliters of drops into a microfluidic device with a wide inlet channel large enough to capture a full field of view (FOV) of drops with a 10× objective. The channel height of the device was 50 micrometers. Brightfield and fluorescence images (Texas RED (TXRED, ex. 540-580 nm, em. 592-669 nm)) of the drops were captured on an inverted epifluorescence microscope (Leica DMi8) with a 10× objective (Leica, NA 0.32). The Leica Application Suite X was used for image acquisition. For each of the eight additive conditions, drops were imaged from three PCR tubes and three FOV per tube (>100 drops per FOV) were captured.

Analysis of endpoint brightfield and fluorescence imaging of drops containing combinations of PCR additives. Drops imaged on the epifluorescence microscope were processed with a custom MATLAB (R2019a) script to measure drop diameter and ROX fluorescence intensity. Measurements of drop diameters D from at least 270 drops per condition were made following thermocycling. All drop measurements were made on the TXRED channel of the acquired images. ROX fluorescence intensities I were normalized by subtracting the background fluorescence IB. Background fluorescence was set as the average pixel value measured from a small (100 px²) background region in the middle of the image using FIJI. The device used for imaging had a channel height of 50 μm, thereby compressing drops larger than 50 μm in diameter and skewing larger drop diameter measurements. Thus, drop diameters greater than this height were adjusted by estimating the compressed drops as oblate spheroids and then using the oblate spheroid volume to calculate the equivalent sphere diameter using the equation

$${D}_{S} = \sqrt[3]{{D}_{E}^{2} h}$$

where D_{S} is the diameter of the equivalent sphere, D_{E} is the measured diameter of a compressed drop, and h is the channel height of 50 micrometers. Normalization of measured D from an initial drop diameter D₀ was determined with the equation (D−D₀)/D₀. The initial D₀ is the average drop diameter measured from drops without PCR additives upon exit from a drop making device as shown in FIG. 14. D₀ was used to normalize the diameter of drops that contained different additives following thermocycling. FIG. 14 illustrates drops at the exit of a drop making device. Initial drop diameter (D₀) is measured as indicated by the dotted line.

Brightfield and fluorescence imaging of drops during thermocycling. For drops imaged before, during, and after thermocycling at each specified cycle, the qPCR machine was paused and one PCR tube was removed to sample drops. Drops were imaged using epifluorescence microscopy to quantify fluorescence from the FAM (6-carboxyfluorescein) dye-labeled TaqMan probe and ROX (6-carboxy-X-rhodamine) reference dye. Five FOV were captured of drops from each tube at each cycle on the epifluorescence microscope for an average of 650 drops after pooling from all FOV (≈0.02% of the drops contained in the PCR tube). The fluorescence intensities of individual drops were measured using a custom MATLAB script 201 (R2019a).

Analysis of brightfield and fluorescence imaging of drops during thermocycling. Drops imaged on the epifluorescence microscope were processed with a custom MATLAB (R2019a) script to measure FAM fluorescence intensity. The FAM fluorescence intensities measured during qRT-PCR were normalized as

$$Δ {R}_{N} / {R}_{N , 0} ,$$

where the qPCR normalized reporter value R_{N} is defined as

$${R}_{N} = {I}_{FAM} / {I}_{ROX} , Δ {R}_{N} = {R}_{N} - {R}_{N , baseline} ,$$

I_{FAM} is the FAM intensity at each cycle, I_{ROX} is the ROX intensity at each cycle, R_{N,baseline} is the average R_{N} value of the first three cycles, and R_{N,0} is the initial R_{N} value.

Virus strains and cell lines. The IAV virus strain used in the present study was A/California/07/2009 (H1N1) (kindly provided by Dr. Christopher Brooke). Viral A/California/07/2009 (H1N1) stocks were propagated and titered on Madin-Darby Canine Kidney (MDCK) cells (kindly provided by Dr. Agnieszka Rynda-Apple). MDCK cells were propagated in DMEM (Corning) media supplemented with 10% fetal bovine serum (HyClone) and 1× Penicillin/Streptomycin (Fisher Scientific). All experimental infections were performed on human alveolar epithelial A549 cells (kindly provided by Dr. Christopher Brooke). A549 cells were propagated in Hams F-12 media (Corning) supplemented with 10% fetal bovine serum and 1× Penicillin/Streptomycin.

Virus infections. A549 cells were seeded into 6-well plates with 1×10⁶ cells per well. The cells were infected with the H1N1 virus at a multiplicity of infection (MOI) of 0.1 in infection media consisting of Hams F-12 supplemented with 1-mM HEPES (HyClone), 1× Penicillin/Streptomycin (Fisher Scientific) and 0.1% BSA (MP Biomedical). Infection with the H1N1 virus was performed in the presence of 1 microgram of TPCK (tolylsulfonyl phenylalanyl chloromethyl ketone)-trypsin/ml (Worthington Biomedical).²⁷ Briefly, cells were washed with 1× Phosphate Buffered Saline (PBS) (Corning) and then incubated with virus that was diluted in infection media for 1 hour. The inoculum was then removed and replaced with fresh infection media and supernatant was collected at 24 hpi.

Screening of qRT-PCR additives to stabilize drops and reduce dye diffusion during thermocycling. To probe how different PCR additives impact drop stability and dye diffusion during thermocycling, the effect of Tween-20, BSA, PEG-6K, and betaine were measured individually and in different combinations in a droplet qRT-PCR assay with 3 wt % (w/w) of PEG-PFPE2 surfactant. To analyze drop stability, drop diameters D were measured post thermocycling from epifluorescence images. To quantify dye diffusion, fluorescence intensities of a thermally stable reference dye (ROX) that is present in all droplet qPCR assays was measured post thermocycling from epifluorescence images. All of the droplet qRT-PCR assays were optimized for TaqMan-based chemistry to detect and quantify in vitro transcribed M gene RNA from IAV. The M gene is a standard target for IAV detection due to its conserved nature across different IAV species. Drops were produced from a solution of qRT-PCR reaction master mix, probe and primers, enzymes and 10⁷ copies/microliters (˜170 cpd) of M gene template. This represents the qRT-PCR reaction mixture with no additives. To test different PCR additives, the qRT-PCR reaction mixture was supplemented with each of the following: BSA, PEG-6K, betaine, Tween-20, Tween-20/PEG-6K, Tween-20/BSA, or Tween-20/BSA/betaine. PEG enhances PCR reactions due to its ability to act as a macromolecular crowding agent, thereby increasing DNA polymerase activity. In drops, PEG has been shown to improve the stability of drops containing high salt content which can occur in standard PCR reaction mix. BSA is a common PCR additive and has been shown previously to limit dye diffusion between drops. Tween-20 has been utilized as an additional surfactant and acts to reduce surface tension in drops while betaine improves PCR amplification of GC rich regions by reducing the formation of nucleic acid secondary structure.

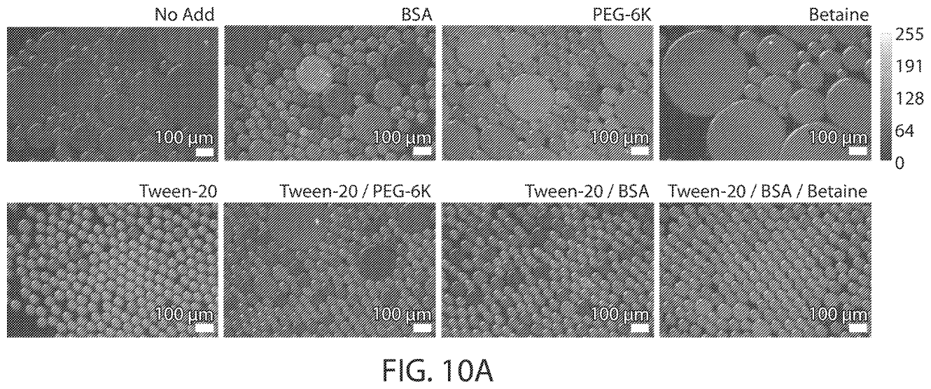

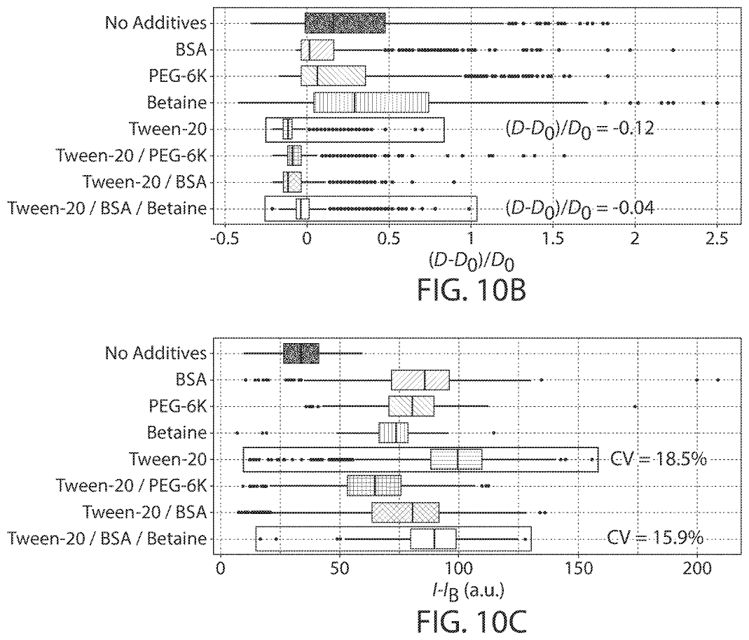

The drops containing 10⁷ copies/microliters of M gene template were thermocycled on a standard qPCR machine with a 30-minute reverse transcriptase step followed by 40 cycles of PCR. Drops were imaged on an epifluorescence microscope following thermocycling in order to assess drop stability from coalescence and retention of the ROX dye. The images of drops that contained no additives, BSA alone, PEG-6K alone, or betaine alone show extensive coalescence with multiple different drop sizes in each image (FIG. 10A). Diameters D were compared to the initial drop diameter D₀ before thermocycling of drops with no PCR additives. Normalized diameters (D−D₀)/D₀ closer to zero indicate less change in drop diameter after thermocycling. The (D−D₀)/D₀ of the various additive combinations in FIG. 10B demonstrate that drops containing no additives, or only a single additive of BSA, PEG-6K, or betaine, had high deviation from zero, a wide distribution of diameters, and a large number of outliers as represented by individual dots. The addition of Tween-20 alone to the PCR reaction mix resulted in a tight distribution of drop diameters. However, (D−D₀)/D₀ was below zero with a value of −0.12, indicating that the Tween-20 drops decreased in size from their initial diameters (FIG. 10B, middle box). As the addition of Tween-20 decreased drop coalescence upon thermocycling, different combinations of the additives were tested with Tween-20. Tween-20 was combined with PEG-6K, BSA and BSA/betaine. The individual additives PEG-6K, BSA, and betaine were chosen as they were utilized in prior droplet PCR assays. By adding Tween-20 to each of these additives, (D−D₀)/D₀ decreased below zero and were found to have lower coefficients of variation (CV) than with no additives and with each of the additives alone without Tween-20 (FIG. 10B). The measured diameters indicate that the addition of Tween-20 greatly decreased both drop coalescence and CV, although epifluorescence images show that Tween-20/PEG-6K and Tween-20/BSA combinations still contained a number of large drops, indicating that the problem of coalescence was not fully resolved. The combination of Tween-20/BSA/betaine drops resulted in (D−D₀)/D₀ closest to zero at −0.04 with very few outliers, indicating these drops were the most stable following thermocycling (FIG. 10B, lower box).

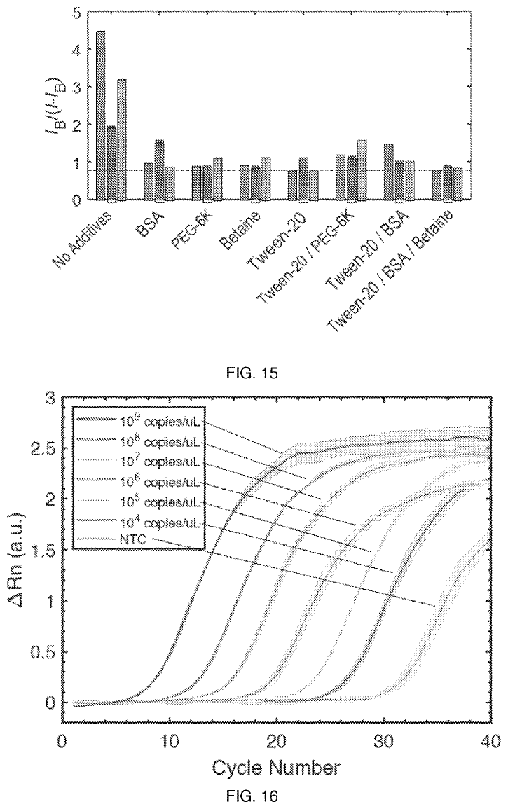

In addition to drop stability, the retention of ROX reference dye within drops after thermocycling was investigated. The fluorescence intensity I of ROX in each of the epifluorescence images was normalized by subtracting the background signal I_{B}. The normalized ROX fluorescence intensity of each drop, I−I_{B}, was measured to quantify diffusion of the dye from the drops (FIG. 10C). A lower value of I−I_{B} indicates that the ROX dye diffused out of the drops and not between neighboring drops. To verify this, the background fluorescence of the oil surrounding the drops was measured and it was found that the background fluorescence intensity was highest for the no additive condition (FIG. 15), indicating that the additives tested here had a positive effect on ROX dye retention in drops during thermocycling.

FIG. 15 depicts mean normalized background fluorescence intensity

$$\frac{{I}_{B}}{I - {I}_{R}}$$

for each PCR additive condition. Bars for each additive represent pooled image data from each qPCR tube sampled. The dashed line is used to show that the Tween-20 and Tween-20/BSA/betaine additive conditions had the lowest normalized background, indicating the best retention of ROX dye in the drops during thermocycling.

A narrow ROX I−I_{B} within drops (smaller CV) is ideal for normalizing the TaqMan probe signal that is produced during thermocycling. In addition, a high I−I_{B} indicates that the dye is not diffusing out of the drops. Thus, drop additives were tested to look for a narrow distribution CV and overall high ROX fluorescence intensity (FIG. 10C). With no additives, I−I_{B} of the ROX dye was 33.6 a.u., the lowest of all eight conditions tested, and indicates a loss of ROX dye from the drops during thermocycling. Based on the data, Tween-20 and the Tween-20/BSA/betaine combination maintained the best drop stability post thermocycling. In terms of ROX dye retention, Tween-20 had the highest I−I_{B} of 99.6 a.u. (FIG. 10C, middle box) compared to Tween-20/BSA/betaine with a I−I_{B} of 89.7 a.u. (FIG. 10C, lower box). However, the Tween-20/BSA/betaine condition resulted in a smaller CV (15.9%) of I−I_{B} than the Tween-20 condition (18.5%). Based on the results, droplet qRT-PCR reactions that contain an additive combination of Tween-20/BSA/betaine have the lowest change in drop diameters following thermocycling with relatively high overall ROX fluorescence intensity and the lowest CV of the fluorescence intensity. Numbers of drops measured and statistical information from FIGS. 10B-10C can be found in Table 1.

TABLE 1

| | | (D-D₀)/D₀ | | | | (I-I_{B}) | | | |
|---|---|---|---|---|---|---|---|---|---|
| | (D-D₀)/D₀ | standard | (D-D₀)/D₀ | D | (I-I_{B}) | standard | (I-I_{B}) | (I-I_{B}) | |
| Additive | mean | deviation | median | CV | mean | deviation | median | CV | N [symbol] |
| No Additives | 0.31 | 0.43 | 0.16 | 33% | 33.6 | 10.4 | 34.0 | 30.9% | 405 |
| BSA | 0.12 | 0.28 | 0.01 | 25% | 83.6 | 20.1 | 85.7 | 24.0% | 915 |
| PEG-6K | 0.25 | 0.40 | 0.09 | 32% | 79.2 | 15.0 | 80.0 | 19.0% | 622 |
| Betaine | 0.46 | 0.60 | 0.29 | 41% | 72.6 | 10.9 | 73.8 | 15.1% | 270 |
| Tween-20 | −0.10 | 0.08 | −0.12 | 9% | 97.0 | 17.9 | 99. [symbol] | 18.5% | 1948 |
| Tween-20/PEG-6K | −0.05 | 0.14 | −0.09 | 15% | 63.9 | 16.9 | 65.3 | 26.4% | 1534 |
| Tween-20/BSA | −0.07 | 0.11 | −0.12 | 12% | 76.2 | 23.5 | 80.5 | 30.8% | 1460 |
| Tween-20/BSA/Betaine | −0.01 | 0.12 | −0.04 | 12% | 88.9 | 14.1 | 89.7 | 15.9% | 1155 |

[symbol]

indicates data missing or illegible when filed

FIGS. 10A-10C show that the addition of additives during PCR thermal cycling Tween-20, BSA, PEG-6K, and betaine were investigated as additives individually and in various combinations to improve drop stability and prevent dye diffusion. FIG. 10A illustrates that drop stability and dye diffusion were measured from epifluorescence images of thermocycled drops with representative images shown of drops with no additives, BSA, PEG-6K, Betaine, Tween-20, Tween-20/PEG-6K, Tween-20/BSA, or Tween-20/BSA/betaine. Fluorescence intensity I is quantified from pixel values ranging from 0 to 255. FIGS. 10B-10C show boxplots of normalized diameter (D-Do)/Do (FIG. 10B) and average ROX fluorescence intensity (FIG. 10C) of drops I−I_{B} following qPCR with different additives. Drops are distributed by quartile, with the vertical line within the box representing the median value and the boxes represent the middle quartile (25%-75% distribution). The lines represent drops in the upper and the lower 25% of the distribution. Dots represent individual drops and extreme values that are either 1.5× larger or smaller than the interquartile range.

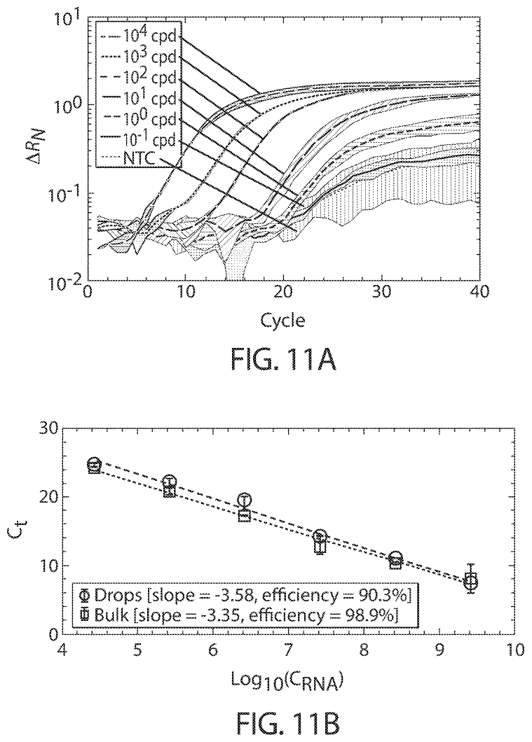

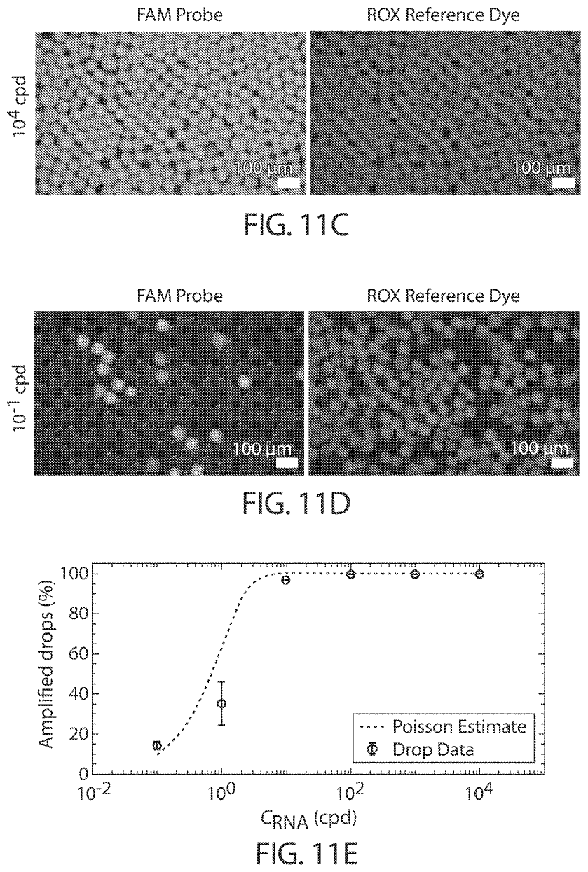

Dilution series demonstrates high efficiency for droplet qRT-PCR. To evaluate the reaction efficiency of the droplet qRT-PCR reaction with the optimized additives Tween-20/BSA/betaine, a dilution series of RNA was performed in drops and as bulk reactions. PCR is based on the principle that amplification begins in an exponential growth phase at early thermocycles before the reaction products become exhausted. This exponential phase corresponds to a reaction efficiency of 100%, where all DNA is multiplied by a factor of two at the completion of every cycle. Desired amplification efficiencies should fall between 90-110%. PCR efficiencies that fall outside of this range will limit dynamic range and sensitivity. A dilution series of a known quantity of target RNA or DNA is required to create a standard curve to relate cycle threshold (CO values to the common logarithm (log 10) of the RNA or DNA concentration. When a standard curve has a slope of −3.33, it indicates that the PCR reaction efficiency is 100%. To perform a standard curve in drops, in vitro transcribed IAV M gene RNA was added as template to the optimized qRT-PCR reaction mix with additives Tween-20/BSA/betaine. Serial RNA dilutions of a range of 10⁴ to 10⁹ copies/microliters in bulk were generated, corresponding to a range of 10⁻¹ to 10⁴ cpd. The amplification curves obtained using qPCR in bulk (FIG. 16) follow the same trend as in drops (FIG. 11A). The Ct values for the dilution series in drops and in bulk were plotted to generate standard curves for both reactions (FIG. 11B). The slopes of the curves yield a droplet reaction efficiency of 90.3% compared to a bulk reaction efficiency of 98.9%, with both falling within the desired 90-110% range (FIG. 11B). It was hypothesized that the lower efficiency in the droplet reactions compared to bulk is due to sequestration of reagents within the drops, resulting in a slightly reduced ability to fully amplify the DNA over 40 cycles before exhaustion of required reaction reagents.

FIG. 16 shows amplification curves of 6 different dilutions of M gene RNA from bulk samples run on a QuantStudio 3 Real-Time PCR System.

An end-point measurement of the drop fluorescence can be performed either with epifluorescence microscopy or custom flow-based detection methods. Here, epifluorescence microscopy was used. Drops from the dilution series in FIG. 11A were imaged on an epifluorescence microscope after 40 PCR cycles. Representative images of FAM and ROX signals in drops containing 10⁴ (FIG. 11C) and 10⁻¹ (FIG. 11D) RNA cpd are shown. As expected, the 10⁴ cpd case resulted in all bright drops due to the high concentration of RNA amplified in each drop, while the 10⁻¹ cpd case resulted in a combination of bright and dark drops due to the limiting dilution of RNA. ddPCR analysis was performed by counting the number of bright versus dark drops from the endpoint reactions of the dilution series. Poisson statistics was used to fit the fraction of positive drops from the dilution series images, following Eq. 1:

λ=−ln(1−p) Eq. 1

where λ is the average copy numbers of target RNA and p is the fraction of positive drops. The percentage of amplified drops demonstrated good agreement with Poisson statistics (FIG. 11E, Table 2). At 10⁻¹ cpd and 10⁰ cpd, there were 14.2% and 38.1% amplified drops, respectively, while at higher concentrations (10¹ through 10⁴ cpd) the number of amplified drops reached 100%, following the Poisson estimate for calculating RNA concentration through ddPCR. The results demonstrate that the optimized qRT-PCR reaction mix maintained drop stability and prevented dye transport between drops, allowing us to normalize the fluorescent signal from the TaqMan FAM probe in amplified drops. In addition, the droplet qRT-PCR exhibited a high PCR reaction efficiency of 90.3%, which was within the desired range of 90-110% for qPCR reactions.

TABLE 2

| | N₊/Nₜₒₜₐₗ | N₊/Nₜₒₜₐₗ |
|---|---|---|
| Dilution Factor | (M gene standards) | (infected supernatant) |
| 10⁰ | 1 | 1 |
| 10⁻¹ | 1 | 0.968 |
| 10⁻² | 1 | 0.771 |
| 10⁻³ | 0.972 | 0.274 |
| 10⁻⁴ | 0.351 | 0.07 |
| 10⁻⁵ | 0.142 | 0.123 |
| Negative Control | 0.065 | 0.082 |

FIGS. 11A-11E show a qRT-PCR dilution series of in vitro transcribed IAV M gene in drops. FIG. 11A depicts amplification curves of six 10-fold dilutions of M gene RNA amplified in drops ranging from 10⁻¹ to 10⁴ copies of RNA per drop (cpd). FIG. 11B shows Cₜ standard curves for the bulk and drop amplification curves. The calculated qRT-PCR reaction efficiency was 90.3% for the drop dilution series and 98.9% for the bulk dilutions series, which falls within the desired range (90% to 110%). FIGS. 11C-11D are representative epifluorescence images of the FAM and corresponding ROX channels of drops containing 10⁴ cpd (FIG. 11C) and 10⁻¹ cpd (FIG. 11D) after 40 thermocycles. FIG. 11E shows droplet digital PCR (ddPCR) analysis displaying the percentage of amplified drops as a function of RNA cpd. The total percentage of amplifying bright drops (circles) increases as a function of RNA concentration and closely follows the Poisson estimate (dotted line).

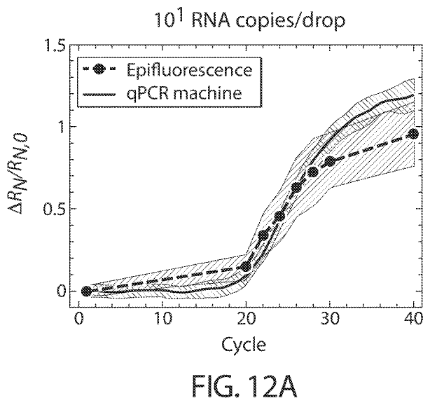

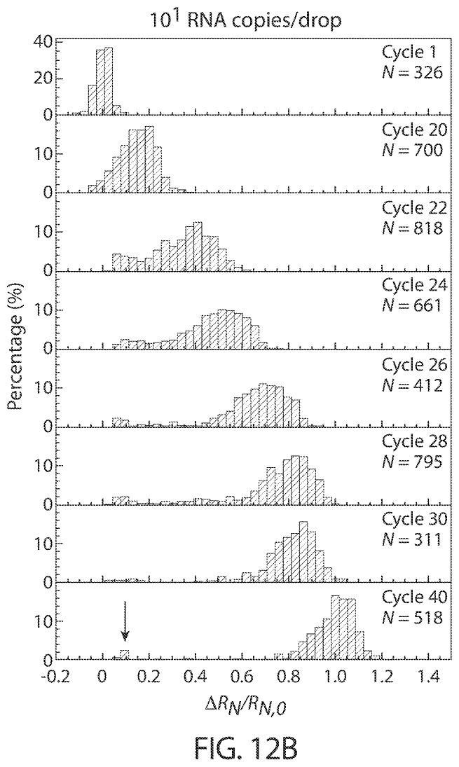

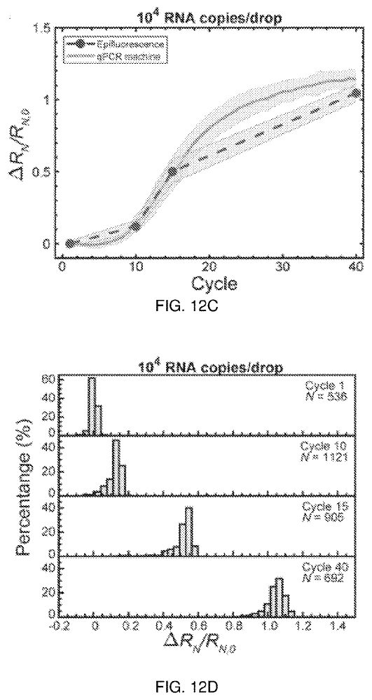

FIGS. 12A-12E show a comparison of droplet qRT-PCR amplification curves generated using a standard qPCR machine and epifluorescence imaging. Drops containing 10¹ M gene RNA cpd (FIG. 12A) were thermocycled using qPCR. The solid green line represents real-time continuous fluorescence measurements of drops at each cycle as determined by qPCR, whereas the blue dashed line represents fluorescence measurements of sampled individual drops at cycle numbers 1, 20, 22, 24, 26, 28, 30, and 40 as determined by epifluorescence microscopy. Shaded error bars represent one standard deviation. FIG. 12B illustrates histograms of individual drop fluorescence values from the epifluorescence images for 10¹ M gene RNA cpd, which show an increase in the fluorescence intensity from cycle 20 to 30. Cycle 1 and 40 represent pre- and post-thermocycling. N represents the number of drops analyzed from multiple epifluorescence images for each cycle. The arrow in cycle 40 represents unamplified drops present in the low RNA loading condition. FIG. 12C depicts 10⁴ M gene RNA cpd that were thermocycled using qPCR. The solid orange line represents continuous fluorescence measurements of drops at each cycle as determined by qPCR, whereas the red dashed line represents fluorescence measurements of individual drops at cycle numbers 1, 10, 15, and 40 as determined by epifluorescence microscopy. Shaded error bars represent one standard deviation. FIG. 12D illustrates histograms of individual drop fluorescence values from the epifluorescence images for 10⁴ M gene RNA cpd, which show an increase in the fluorescence intensity from cycle 15 to 40. Cycle 1 and 40 represent pre- and post-thermocycling. N represents the number of drops analyzed from multiple epifluorescence images for each cycle. (E) Changes in fluorescence as cycle numbers increase are observed from representative epifluorescence images of 10¹ or 10⁴ M gene RNA cpd at cycles 22 and 28 and cycles 10 and 15, respectively.

Generation of droplet PCR amplification curves from direct qRT-PCR thermocycling compared to epifluorescence microscopy. Concentration of nucleic acid present within a sample can be determined using qRT-PCR. In the prior section, a novel method was introduced for testing qRT-PCR additives to obtain amplification curves across five orders of magnitude of M gene RNA in drops. Here, a high-resolution investigation of two concentrations of M gene in drops undergoing real-time PCR amplification was performed. Drops containing low (10¹ cpd) and high (10⁴ cpd) viral RNA were studied by sampling drops at various cycle numbers on the qRT-PCR machine and imaging under epifluorescence microscopy. Real-time PCR amplification curves were obtained from the thermocycled drops in the qPCR machine and compared these curves to constructed amplification curves arising from hundreds of individual drops imaged using epifluorescence microcopy.

Amplifying RNA within drops on a standard qPCR machine generated amplification curves of all the drops contained within the sample tube, approximately 300,000 drops. The qPCR machine tracks fluorescence amplification of all drops in the tube at each cycle number between 0 to 40, which is called continuous data. Hundreds of randomly sampled drops imaged on an epifluorescence microscope over multiple thermocycles were sampled, which is called discontinuous data. This allowed for the construction of amplification curves using microscopy that resembled the real-time continuous measurements performed on the standard qPCR machine. Thermocycling drops within the qPCR machine without interruption generated a continuous amplification curve (FIG. 12A, solid line) and is representative of a bulk sample (FIG. 16). By contrast, a discontinuous amplification curve was produced by imaging drops from individual tubes at specific cycles on an epifluorescence microscope (FIG. 12A, dashed line). Drops from the low RNA loading condition were analyzed across eight cycles (cycle 1, 20, 22, 24, 26, 28, 30, and 40) (FIG. 12A). Cycle 1 provides a baseline fluorescence measurement. At cycle 20, the amplification curve begins to increase exponentially. Drops are then imaged after every two cycles from cycle 20 to 30 to capture the exponential region of the amplification curve. A final endpoint measurement is taken at cycle 40. The normalized fluorescence data from the epifluorescence images (FIG. 12A, blue dashed line) are plotted for each cycle measured and produce a curve similar to that of the qPCR machine (FIG. 12A, solid line), validating that an amplification curve can be constructed from the discontinuous epifluorescence data. The distributions of drop fluorescence from epifluorescence images at cycles 1, 20, 22, 24, 26, 28, 30, and 40 for the low RNA loading condition are presented in FIG. 12B. As amplification starts to increase at cycle 20, the distribution of drops shifts to the right as the fluorescence increases. From cycle 20 to 30, it was observed that a majority of the drops began to amplify while a small population did not (FIG. 12B, arrow, cycle 40), forming a bimodal distribution. It was hypothesized that this small population of non-amplifying drops did not contain template RNA. This would indicate that the 1.7×10¹ cpd loading was an overestimate as empty drops are not expected at this concentration. In comparison, the high RNA loading condition (1.7×10⁴ cpd) should result in every drop containing RNA, with all drops amplifying in unison, and should not result in a bimodal distribution as observed with the low RNA loading condition. To test this, drops containing 1.7×10⁴ RNA cpd were imaged at 0, 10, 15 and 40 cycles (FIG. 12C). As with the low RNA loading condition, the normalized fluorescence data from the high RNA loading condition epifluorescence images produced a curve (FIG. 12C, dashed line) similar to that of the qPCR machine (FIG. 12C, solid line). The distributions of drop fluorescence at each cycle for the high RNA loading condition are presented in FIG. 12D.

As discrete particles such as RNA are encapsulated into drops following a Poisson distribution, it was predicted that the high RNA loading condition (1.7×10⁴ cpd) would create a tight distribution (CV=0.77%) while the low RNA loading condition (1.7×10¹ cpd) would create a wide distribution (CV=24.3%) of RNA between drops, respectively. The CV is calculated as

$${λ}^{- \frac{1}{2}} ,$$

as defined by the Poisson distribution, where λ is the RNA loading in cpd. The high RNA loading condition resulted in all drops amplifying as predicted. In addition, the fluorescence intensities of the high loading RNA condition showed tighter distributions at all cycles compared to the amplified drops in the low RNA loading conditions. Representative images of drops for both 1.7×10¹ and 1.7×10⁴ cpd at early and late cycles provide a visual reference for the distributions in the histograms (FIG. 12E). The similar trend of drop measurements between the qPCR machine and the epifluorescence images demonstrates that RNA concentrations in individual drops can be extrapolated from real-time PCR amplification curves produced by either standard qPCR thermocycling or epifluorescence microscopy. These methods can therefore be utilized to determine nucleic acid concentrations from a large population of drops without the need for custom flow-based methods.

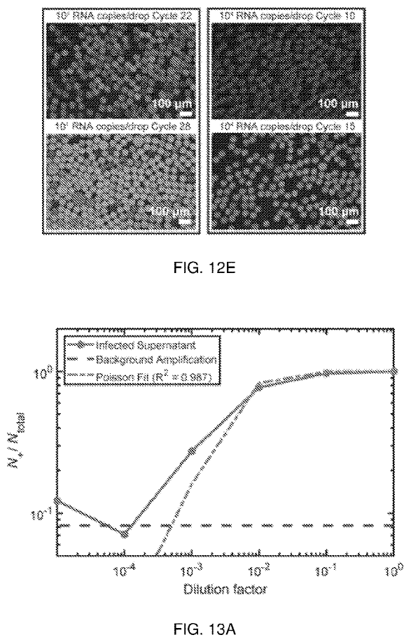

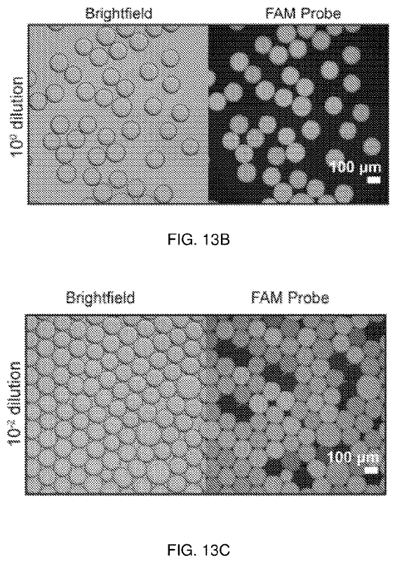

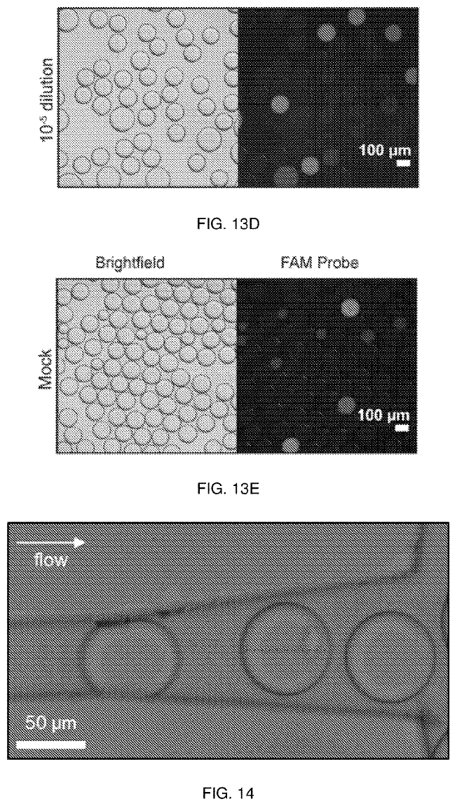

qRT-PCR amplification and quantification of live influenza A virus. Following successful amplification of in vitro transcribed RNA, the optimized droplet qRT-PCR assay was applied to quantify the number of IAV genomes present in the supernatant from infected cells using ddPCR analysis. A549 cells, a human alveolar epithelial cell line, was infected with the A/California/07/2009 (H1N1) IAV at a low MOI of 0.1. At 24 hours post infection, six dilutions of supernatant were prepared from infected cells starting with undiluted, or 10⁰ dilution, down to a 10⁻⁵ dilution. Supernatant from mock infected cells was included as a control to determine the level of background amplification and the detection limit for live virus. Infected supernatant dilutions and undiluted supernatant from mock infected cells were added to the optimized qRT-PCR master mix and drops were produced, thermocycled, and analyzed on the epifluorescence microscope. In this case, the virus was heat lysed during the reverse transcription step at 60° C. for 30 minutes and then amplified without an RNA extraction step. This direct PCR method allowed us to perform ddPCR directly from the supernatant of infected cells to quantify the amount of RNA present. The fraction of positive drops to total drops (N₊/Nₜₒₜₐₗ), also defined asp in Eq. 1, was measured and plotted as a function of dilution factor (FIG. 13A, solid line). A Poisson fit with an R² value of 0.987 was used to estimate the average number of JAY genomes per drop at each dilution (FIG. 13A, dot-dashed line, Table 2). The undiluted supernatant was estimated to contain 175 cpd, corresponding to 3.34×10⁶ copies/microliters. This value was compared to the measured concentration of the undiluted supernatant obtained using qRT-PCR. The Ct value of the undiluted supernatant resulted in a concentration of 1.96×10⁶ copies/microliters, based upon the standard curve from FIG. 11A, in agreement with the Poisson estimate. The supernatant dilutions (FIG. 13A, solid line) followed the Poisson fit down to the 10⁻³ dilution (0.175 cpd). The background amplification level of mock infected cells was 0.082 cpd (FIG. 13A, dashed line). Representative images are shown and demonstrate that undiluted supernatant results in every drop amplifying after 40 cycles (FIG. 13B). In comparison, a 10⁻² dilution of supernatant, which was estimated to contain 1.75 cpd, results in a majority of drops amplifying (77%) with a few that remain dark (FIG. 13C). The lowest 10⁻⁵ dilution of supernatant from infected cells is similar to supernatant from mock infected cells and is therefore representative of background amplification (FIGS. 13D and 13E). The limit of detection was reached at a 10⁻³ dilution level, in which N₊/Nₜₒₜₐₗ was measured to be 0.320 cpd. This demonstrates the ability of the droplet qPCR assay to detect viral RNA down to a single cpd. Thus, the ability to amplify and quantify JAY RNA directly from infected cells across four orders of magnitude, down to single viral genome copies encapsulated within drops is demonstrated herein.

FIGS. 13A-13E show JAY amplified using droplet qRT-PCR method. FIG. 13A shows that the supernatant from infected cells was diluted and combined in drops with the optimized PCR mixture. The fraction of amplified drops is measured as the fraction of the number of fluorescent drops (N₊) and the total number of drops (Nₜₒₜₐₗ). A ddPCR analysis of end point epifluorescence imaging shows the dilution series of viral RNA in drops (solid line) coincides with the Poisson fit (dot-dashed line). Detection of viral RNA was achieved over four orders of magnitude before reaching the level of background amplification as determined using supernatant from mock infected cells (dashed line). FIGS. 13B-13E show representative epifluorescence images of thermocycled drops containing various dilutions of viral supernatant from infected cells, including a mock sample with no virus.

In this work, a droplet qRT-PCR assay is presented for quantifying IAV following systematic testing of different PCR additives, resulting in the optimal combination of Tween-20/BSA/betaine to maintain drop stability and limit dye diffusion. The ability to use a standard qPCR machine to generate real time amplification curves of hundreds of thousands of drops is demonstrated herein for the first time. This data is correlated with hundreds of drops sampled using epifluorescence microscopy. Measurements of real-time PCR amplification in drops using a standard qPCR machine or epifluorescence microscope can eliminate the need for complicated custom microfluidic thermocycling devices. As qPCR machines and epifluorescence microscopes are common instruments in many laboratories, this method can be used to systematically test droplet qPCR additives for quantifying genomes within a large population of drops.

A wide concentration range of both in vitro transcribed viral RNA and viral supernatant from infected cells were tested in drops. The optimized droplet qPCR assay enabled quantification of 10⁻¹ to 10⁴ cpd of in vitro transcribed IAV M gene. To further demonstrate the utility of this method, viral IAV genomes were quantified from infected cells. Using ddPCR, IAV RNA was measured over four orders of magnitude down to 0.320 cpd, or a single viral genome per drop. This demonstrates the high sensitivity and precision of the droplet qPCR assay. Importantly, this work establishes the ability to directly amplify viral RNA without the need for RNA extraction and with very little reagent, which is advantageous in challenging resource-limited situations such as the COVID-19 pandemic. It is believed that these findings will have great impact in future studies for accurately quantifying viral genomes using droplet qPCR.

### Example 5

The COVID-19 pandemic led to unprecedented lockdowns throughout much of the world. An informed lifting of restrictions will rely on a robust system of testing and monitoring if resurgence is to be avoided. Success will depend upon development of rapid tests for two disease indicators, virus and antibody, which provide important data to inform public health measures and optimize resource allocation. Quantitative detection of virus identifies asymptomatic cases characteristic of COVID-19 and antibody detection identifies immunity. Point-of-care microfluidic solutions are pursued for detection of both antigen and antibody. A rapid, highly sensitive method for virus detection is presented herein, using droplet digital RT-LAMP amplification. This method uses orders of magnitude less reagent, heat lysis to access the viral RNA, eliminating costly RNA extraction steps, and has potential to detect down to single virions in tiny, picoliter volumes.

The gold-standard method for detection of SARS-CoV-2 is quantitative reverse transcription PCR (qRT-PCR). Currently, each qRT-PCR test requires significant reagent consumption, specialized equipment, trained operators, and several hours to perform. Nearly all COVID-19 case data collected so far has been using conventional qRT-PCR. However, in the coming months, expanding this level of surveillance across a global scale will call for new strategies that conserve limited reagents, decrease turnover time, and open avenues for point-of-care monitoring. To this end, the method disclosed herein eliminates the need for sophisticated fluorescent detection, reduces reagent usage by orders of magnitude, and takes under 30 minutes to complete. Furthermore, data collection and analysis may be accomplished using a smartphone camera and software.

The 2019 emergence of the novel coronavirus SARS-CoV-2 has led to a wave of infections worldwide medically characterized by and culminating in potentially deadly bilateral pneumonia, a condition known as COVID-19. As the pandemic continues, mitigation strategies have primarily taken the form of universal quarantines resulting in significant economic damage and the indefinite suspension of public services. In order to mount a measured response to the highly transmissible SARS-Cov-2, it is becoming widely accepted that frequent diagnostic testing for both virus and antibodies would allow the already immune to continue operating economically and socially, while the infected and those at most risk can remain safely isolated. While tracking the immune response using serological surveying is essential to understanding the spread of SARS-CoV-2, recent attention has been focused on the potential for individuals to continue transmitting the virus, even after recovery, a condition that cannot be detected using immunological testing alone. Current methods for the detection of viral RNA itself has taken the form of RT-PCR, a gold standard method, but costly in terms of reagent consumption and the need for expensive, specialized equipment. This calls for the development of technologies that can perform both rapid and high-throughput detection of viral expression, while also conserving precious reagents, predicted to soon be in short supply.

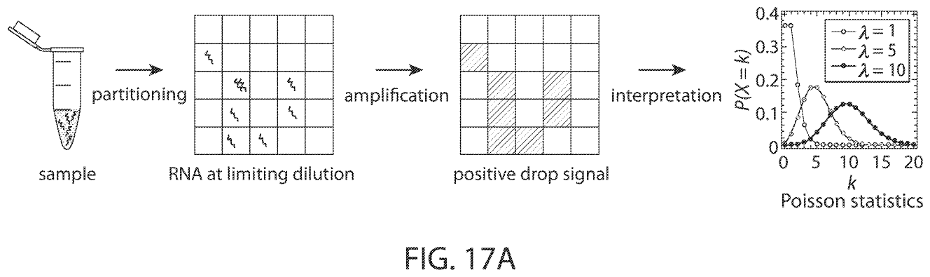

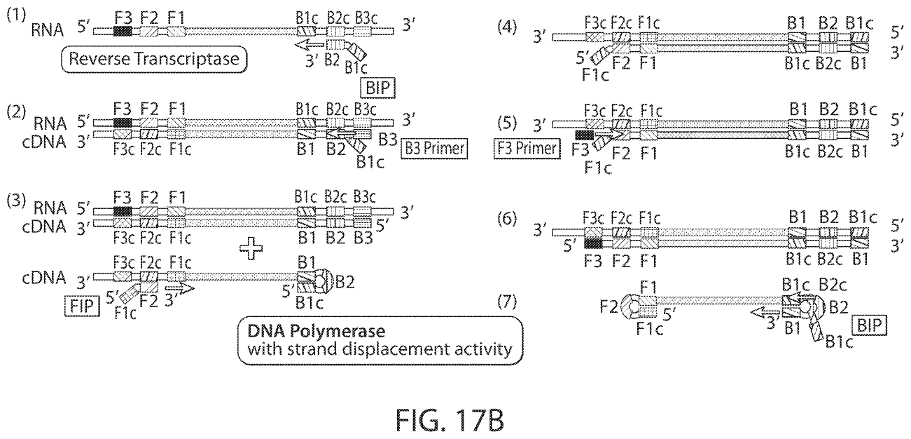

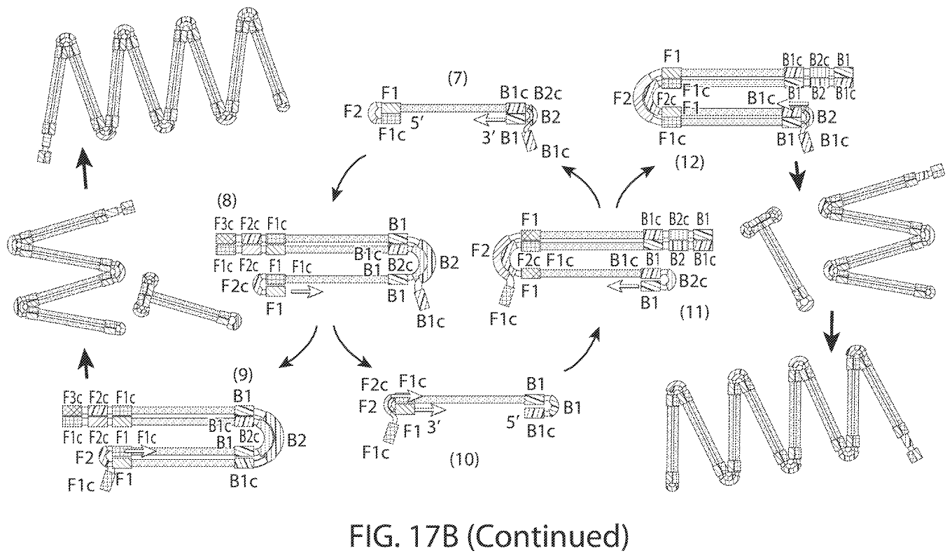

Digital drop PCR (ddPCR) has emerged as a quantitative method of RNA detection based on well established digital methods. Partitions can be achieved in a variety of ways but when performed entirely within picoliter volumes of drops the perceived test spot sizes and volumes are tunable and with the potential for single molecule detection. The method is quantitative, potentially leading to results similar in accuracy to RT-PCR and relies on a straightforward statistical analysis to reach conclusions regarding the amount of RNA present in the original sample. The technique has already begun being used to detect SARS-CoV-2. Samples (FIG. 17A, sample) are diluted to vanishing concentration and, along with reagents, confined within individually stabilized emulsion droplets of 10s to 100s of μm in diameter in a process called partitioning. Partitioned drops are amplified and DNA is detected using intercalating, pH sensitive, or TaqMan reagents. Dilution results in a large number of drops that were not loaded with any viral RNA, resulting in no color change. Droplet loading is governed by well-characterized Poisson distributions, and statistical analysis can be performed on the binary outcome. Briefly summarized, binomial probability p of whether a viral RNA target enters a drop is given by p=1−(1−1/n)^{m} where m represents molecules in drops and n is the total number of drops partitioned. In the limit of large m, the term (1−1/n) can be approximated by e^{-1/n}. Substituting the average value λ=n/m, the probability is described by a Poisson distribution leading to λ=−ln(1−m/n) where m represents positive partitions (FIG. 17A, Poisson statistics). Poisson statistics permit the prediction of RNA copy numbers simply by knowing the ratio of positive to negative drops through the relationship C=λ/V_{d} where C is sample concentration and V_{d} is partition (drop) volume.

FIGS. 17A-17B are schematics of the dPCR and RT-LAMP processes. FIG. 17A shows a target sample divided into compartments, confining RNA at limiting dilution. Amplification results in positive drop signals indicated by color change. Poisson statistics are used to interpret the original copy number in the sample. FIG. 17B illustrates a summary of the one-pot RT-LAMP isothermal RNA amplification method.

Traditional methods of PCR and the gold standard techniques of RT-qPCR have been developed for SARS-CoV-2 detection, are difficult to implement in point-of-care devices as temperature cycles reach upwards of 95° C. and upwards of 40 cycles. Circumventing these difficulties is possible using isothermal amplification methods of reverse transcription-loop-mediated isothermal amplification (RT-LAMP), technique requiring a single, lower temperature with no cycling, highly amendable as a point-of-care alternative to PCR. The technique has been shown to detect MERS-CoV and SARS-1. The technique uses four primers, which have been identified for amplification of SARS-CoV-2 RNA, with some methods already implemented within 30 minutes. The inner strand primers are called FIP and BIP which synthesize new DNA strands. The outer primers are called BP and FP and anneal to the template strand. It is a one-pot method where amplification occurs at a fixed temperature of between 60 and 65° C. This significantly simplifies any hardware needed for performing viral detection, even simple handwarmers have been shown to work effectively in a field deployment scenario. Furthermore, a variety of colorful chromogenic molecules indicate when RNA has been sufficiently amplified, making naked eye operation a possibility, conditions that satisfy the CLIA waiver requirements.

The technique can work with a variety of sample collection methods. An RNA extraction step, typically using expensive and soon-to-be vanishing commercial reagent kits, may be circumvented by a heating step at the same temperature as RT-LAMP. Nucleic acid amplification tests have been compared to determine best performance and the COVID-19 RT-qPCR assay has been evaluated.

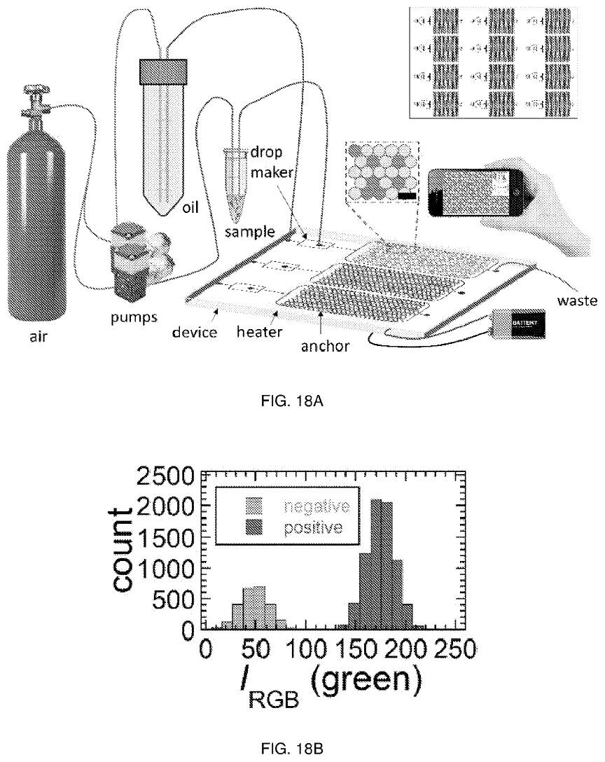

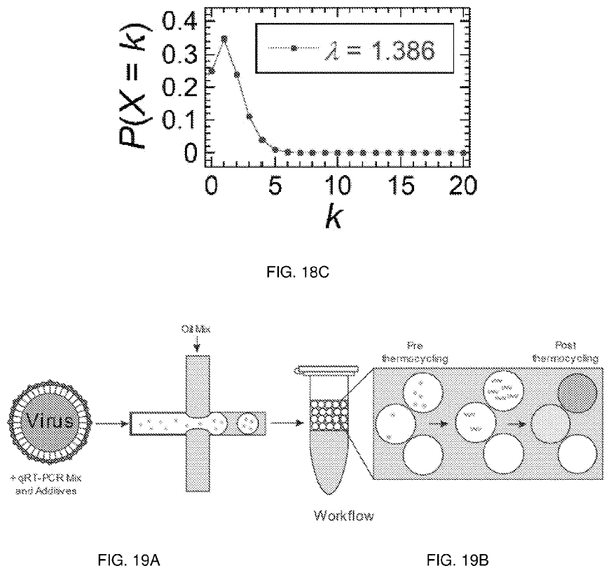

Integrating ddPCR with RT-LAMP on a microfluidic chip provides a rapid diagnostic for detecting SARS-CoV-2 viral RNA. Soft microfluidics are used to fabricate microscale channels in polydimethylsiloxane (PDMS) bonded to a glass slide (FIG. 18A, device). Pressure is provided by ordinary compressed air (FIG. 18A, air) and regulated using small membrane pumps (FIG. 18A, pumps). A drop maker (FIG. 18A, drop maker) partitions RT-LAMP reagents and swabbed samples at limiting dilutions (FIG. 18A, sample) separated and stabilized by fluorinated surfactant solution in oil (FIG. 18A, oil). Flow traps and other methods have been used to fix partitions in the plane of the chip for recording. Drops are trapped in surface tension anchors (FIG. 18A, anchor) creating a hexagonal grid of thousands of RT-LAMP reactions. A battery powered transparent layer of indium tin oxide (ITO) provides heat for RT-LAMP reactions (FIG. 18A, heater) offering an elegant simplification to bulkier heater implementations. After a period of time, an ordinary camera phone captures positive (green) drops and performs. It has been demonstrated that RBG image analysis can capture the intensities of LAMP chromogenic reagents in drop volumes. Poisson statistics to calculate the RNA concentration in the original sample. After analysis is complete, the test drops can be flushed into waste (FIG. 18A, waste) and reused for another sample. An array of 12 devices of 1072 anchors can fit on a microscope slide (FIG. 18A, inset).

Reagent consumption is reduced by orders of magnitude and for even greater automation, results can be recorded and interpreted using smartphone camera and software. An app guides the photography of the test drop array, fitting the final captured image into a calibrated mask associated with the spacing of the anchor array. The RGB colors of the image are analyzed and positive and negative drops identified based upon RGB intensity values (FIG. 18B). RNA copy number present in the original sample is calculated using a Poisson fit (FIG. 18C).

FIGS. 18A-18C illustrate the design and workflow of the ddPCR device for detection of SARS-CoV-2 RNA using RT-LAMP. FIG. 18A is a schematic of device operation. Air is supplied to two small membrane pumps that pressurize and surfactant/oil and sample reservoir sending them to the drop maker on the device. Drops are collected into the viewing area and held in a hexagonal grid using special drop anchors printed onto the device. A battery operated ITO heating element provides 60° C. heating to drive the RT-LAMP reaction. After amplification, the drops are photographed and analyzed using smartphone camera and software. Inset: zoom of hexagonal grid of positive and negative drops. Scale bar is 300 micrometers. FIG. 18B shows an example of histogram plot of green RGB channel intensity providing software count of positive and negative drops. Total drops counted number ≈10,000. FIG. 18C shows that using Poisson statistics, it is possible to identify a distribution of copy numbers from drop and a quantitative estimate of original sample copy number.

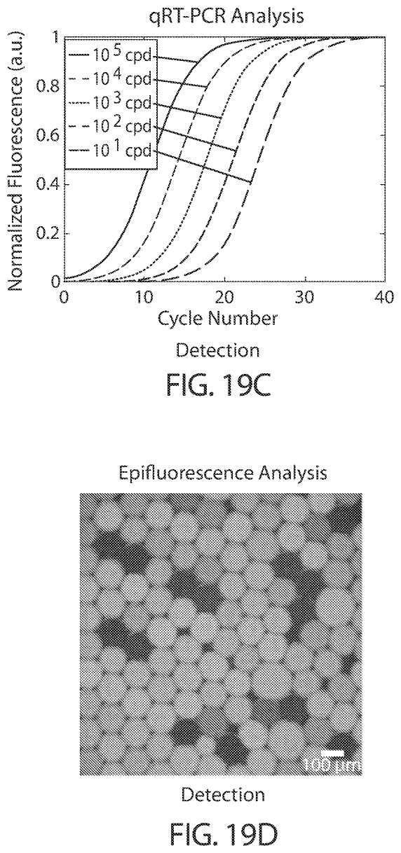

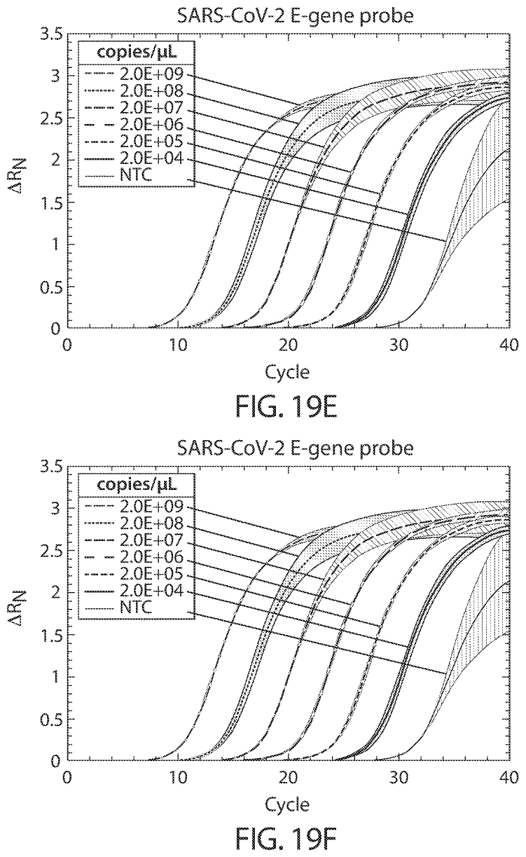

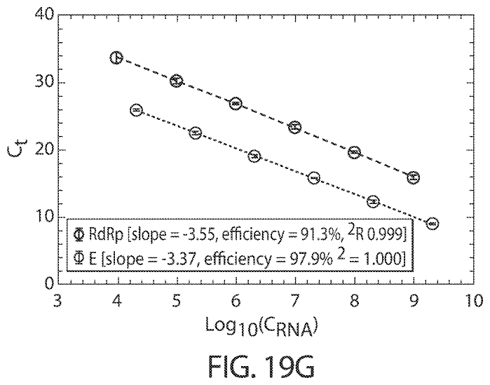

FIGS. 19A-19G show example workflow and quantitative detection of SARS-CoV-2 E and RdRp genes in drops. Rapid detection and assaying of influenza viruses using microfluidics (FIGS. 19A-19D) was applied to perform rapid detection of SARS-CoV-2 and antibodies to the virus. The virus was cultured in BSL-3 and data demonstrates amplification of SARS-CoV-2 RdRp and E segments using qRT-PCR (FIGS. 19E-19F). The rapid assays were validated using positive and negative clinical samples. These results show that these genes can be amplified using qPCR (FIGS. 19E-19G) and are adaptable to drop-based technology. Primers necessary to perform RT-LAMP allow for isothermal amplification without thermocycling. LAMP primers were selected to develop RT-LAMP. and RT-LAMP was demonstrated on RdRP and E in vitro transcribed RNA of SARS-CoV-2. RT-LAMP was also demonstrated in drops of in vitro transcribed RdRP and E RNA. RT-LAMP of SARS-CoV was performed in a BSL-3 laboratory and the SARS-CoV-2 was amplified, quantified, and visualized RT-LAMP of SARS-CoV-2 was demonstrated in BSL-3 facility. RT-LAMP of SARS-CoV-2 was demonstrated in drops BSL-3 facility.

A point-of-care device was created that is optimized with spiked concentrations of virus that correspond to viral concentrations expected from a nasopharyngeal swab sample. The device is 100× more sensitive compared to qPCR, which has an approximate LOD of 1×10³ copies/mL. The device is battery-powered and requires a small air tank to transfer drops onto the device. Results can be read out by a cell phone camera (FIG. 18A). A simple microfluidic chip was fabricated on ITO glass to heat drops at 60 and 65° C. Pressure setup was integrated on chip. On-chip isothermal amplification of spiked SARS-CoV-2 was demonstrated in drops. The Limit of Detection using drops spiked with virus dilutions was also determined.

The point-of-care is field deployed to detect virus from nasal swab solution directly onto the device. Positive and negative samples were obtained to verify the assay. The Limit of Detection (LOD) using drops spiked with virus dilutions was determined. Testing was deployed for analyzing nasopharyngeal swabs. This method is rapid, does not require RNA extraction kits, uses little reagent, if reagent limitation becomes an issue.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the disclosure includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method, comprising:
producing droplets containing a sample within a microfluidic device;
amplifying nucleic acid suspected of being in the droplets using loop mediated isothermal amplification; and
determining fluorescence of the droplets.

2. The method of claim 1, comprising producing at least 10⁵ droplets.

3. The method of any one of claim 1 or 2, comprising producing at least 10⁶ droplets.

4. The method of any one of claims 1-3, wherein the sample arises from a human.

5. The method of any one of claims 1-4, wherein the sample comprises saliva.

6. The method of any one of claims 1-5, wherein the sample arise from a nose swab.

7. The method of any one of claims 1-6, comprising heating the sample to at least 65° C. while amplifying the nucleic acid.

8. The method of any one of claims 1-7, comprising determining fluorescence of the droplets within 10 minutes of partitioning the sample into the droplets.

9. The method of any one of claims 1-8, comprising introducing primers into the droplets prior to amplifying nucleic acid.

10. The method of claim 9, wherein the primers comprise RT-LAMP primers.

11. The method of any one of claims 1-10, wherein the nucleic acid comprises RNA.

12. The method of any one of claims 1-11, wherein the nucleic acid arises from a virus.

13. The method of any one of claims 1-12, wherein the nucleic acid arises from COVID-19.

14. The method of any one of claims 1-13, wherein the nucleic acid arises from a SARS virus.

15. The method of any one of claims 1-14, wherein the nucleic acid arises from a MERS virus.

16. The method of any one of claims 1-15, wherein the nucleic acid arises from a bacteria.

17. The method of any one of claims 1-16, comprising amplifying the nucleic acid to produce at least 10⁹ copies.

18. A method, comprising:
mixing a saliva sample suspected of containing a target nucleic acid with an aqueous fluid to form a mixture, wherein the aqueous fluid comprises amplification reagents;
forming a plurality of at least 10⁵ droplets of the mixture, contained within a carrier fluid;
causing amplification of the target nucleic acid to produce amplified nucleic acids, using the amplification regents, within the plurality of droplets;
determining droplets of the plurality of droplets that contain the amplified nucleic acids; and
determining a nucleic acid concentration in the saliva sample based on the determination of the droplets containing the amplified nucleic acids.

19. The method of claim 18, comprising forming a plurality of at least 10⁶ droplets of the mixture.

20. The method of any one of claim 18 or 19, wherein the sample arises from a human.

21. The method of any one of claims 18-20, comprising heating the plurality of droplets to at least 65° C. while amplifying the target nucleic acid.

22. The method of any one of claims 18-21, wherein the amplification regents comprise primers.

23. The method of claim 22, wherein the primers comprise RT-LAMP primers.

24. The method of any one of claims 18-23, wherein the target nucleic acid comprises RNA.

25. The method of any one of claims 18-24, wherein the target nucleic acid comprises DNA.

26. The method of any one of claims 18-25, wherein the target nucleic acid arises from a virus.

27. The method of any one of claims 18-26, wherein the target nucleic acid arises from COVID-19.

28. The method of any one of claims 18-26, wherein the target nucleic acid arises from a SARS virus.

29. The method of any one of claims 18-26, wherein the target nucleic acid arises from a MERS virus.

30. The method of any one of claims 18-25, wherein the target nucleic acid arises from a bacterium.

31. The method of any one of claims 18-30, comprising causing amplification of the target nucleic acid to produce at least 10⁹ copies.

32. A method of determining an attomolar concentration of a species in a fluid, the method comprising:
providing an aqueous fluid having a volume of at least 1 ml and containing a species at a concentration of less than 1 fM;
forming a plurality of at least 10⁵ droplets of the aqueous fluid, contained within a carrier fluid;
determining droplets of the plurality of droplets that contain the species; and
determining a concentration of the species in the aqueous fluid based on the determination of droplets containing the species.

33. The method of claim 32, comprising forming a plurality of at least 10⁶ droplets of the mixture.

34. The method of any one of claim 32 or 33, wherein the aqueous fluid arises from a human.

35. The method of any one of claims 32-34, wherein the aqueous fluid comprises saliva.

36. The method of any one of claims 32-35, wherein the aqueous fluid arises from a nose swab.

37. The method of any one of claims 32-36, wherein the species is a nucleic acid.

38. The method of claim 37, further comprising amplifying the nucleic acid to produce amplified nucleic acids within the plurality of droplets.

39. The method of claim 38, comprising heating the plurality of droplets to at least 65° C. while amplifying the nucleic acid.

40. The method of any one of claim 38 or 39, further comprising introducing amplification reagents into the droplets.

41. The method of any one of claims 38-40, comprising amplifying the nucleic acid to produce at least 10⁹ copies.

42. The method of any one of claims 38-41, comprising introducing amplification reagents into the droplets during formation of the droplets.

43. The method of any one of claims 38-42, comprising introducing amplification reagents into the droplets after formation of the droplets.

44. The method of any one of claims 38-43, wherein the amplification regents comprise primers.

45. The method of claim 44, wherein the primers comprise RT-LAMP primers.

46. The method of any one of claims 37-45, wherein the nucleic acid comprises RNA.

47. The method of any one of claims 37-46, wherein the nucleic acid comprises DNA.

48. The method of any one of claims 37-45, wherein the nucleic acid arises from a virus.

49. The method of any one of claims 37-45, wherein the nucleic acid arises from COVID-19.

50. The method of any one of claims 37-45, wherein the nucleic acid arises from a SARS virus.

51. The method of any one of claims 37-45, wherein the nucleic acid arises from a MERS virus.

52. The method of any one of claims 37-46, wherein the nucleic acid arises from a bacterium.

53. A method of detecting a species at an attomolar concentration, the method comprising:
providing an aqueous fluid having a volume of at least 1 ml and containing a species at a concentration of less than 1 fM;
forming a plurality of at least 10⁵ droplets of the aqueous fluid, contained within a carrier fluid; and
determining droplets of the plurality of droplets that contain the species, wherein no more than 10 droplets per 10⁵ droplets contain the species.

54. The method of claim 53, comprising forming a plurality of at least 10⁶ droplets of the mixture.

55. The method of any one of claim 53 or 54, wherein the aqueous fluid arises from a human.

56. The method of any one of claims 53-55, wherein the aqueous fluid comprises saliva.

57. The method of any one of claims 53-56, wherein the aqueous fluid arises from a nose swab.

58. The method of any one of claims 53-57, wherein the species is a nucleic acid.

59. The method of claim 58, further comprising amplifying the nucleic acid to produce amplified nucleic acids within the plurality of droplets.

60. The method of claim 59, comprising heating the plurality of droplets to at least 65° C. while amplifying the nucleic acid.

61. The method of any one of claims 59-60, further comprising introducing amplification reagents into the droplets.

62. The method of any one of claims 59-61, comprising amplifying the nucleic acid to produce at least 10⁹ copies.

63. The method of any one of claims 59-62, comprising introducing amplification reagents into the droplets during formation of the droplets.

64. The method of any one of claims 59-63, comprising introducing amplification reagents into the after during formation of the droplets.

65. The method of any one of claims 59-64, wherein the amplification regents comprise primers.

66. The method of claim 65, wherein the primers comprise RT-LAMP primers.

67. The method of any one of claims 58-66, wherein the nucleic acid comprises RNA.

68. The method of any one of claims 58-67, wherein the nucleic acid comprises DNA.

69. The method of any one of claims 58-66, wherein the nucleic acid arises from a virus.

70. The method of any one of claims 58-66, wherein the nucleic acid arises from COVID-19.

71. The method of any one of claims 58-66, wherein the nucleic acid arises from a SARS virus.

72. The method of any one of claims 58-66, wherein the nucleic acid arises from a MERS virus.

73. The method of any one of claims 58-66, wherein the nucleic acid arises from a bacterium.

74. The method of any one of claims 53-73, wherein no more than 1 droplet per 10⁵ droplets contain the species.

75. The method of any one of claims 53-74, wherein no more than 1 droplet per 10⁶ droplets contain the species.

76. A method of detecting a target nucleic acid in saliva, comprising:
mixing a saliva sample suspected of containing a target nucleic acid with an aqueous fluid to form a mixture, wherein the aqueous fluid comprises amplification reagents;
forming a plurality of at least 10⁵ droplets of the mixture, contained within a carrier fluid;
causing amplification of the target nucleic acid to produce amplified nucleic acids, using the amplification regent, within the plurality of droplets; and
determining droplets of the plurality of droplets that contain the amplified nucleic acids, wherein no more than 10 droplets per 10⁵ droplets contain the amplified nucleic acids.

77. The method of claim 76, comprising forming a plurality of at least 10⁶ droplets of the mixture.

78. The method of any one of claim 76 or 77, wherein the sample arises from a human.

79. The method of any one of claims 76-78, comprising heating the plurality of droplets to at least 65° C. while amplifying the target nucleic acid.

80. The method of any one of claims 76-79, wherein the amplification regents comprise primers.

81. The method of claim 80, wherein the primers comprise RT-LAMP primers.

82. The method of any one of claims 76-81, wherein the target nucleic acid comprises RNA.

83. The method of any one of claims 76-82, wherein the target nucleic acid comprises DNA.

84. The method of any one of claims 76-83, wherein the target nucleic acid arises from a virus.

85. The method of any one of claims 76-84, wherein the target nucleic acid arises from COVID-19.

86. The method of any one of claims 76-84, wherein the target nucleic acid arises from a SARS virus.

87. The method of any one of claims 76-84, wherein the target nucleic acid arises from a MERS virus.

88. The method of any one of claims 76-83, wherein the target nucleic acid arises from a bacterium.

89. The method of any one of claims 76-88, comprising causing amplification of the target nucleic acid to produce at least 10⁹ copies.

90. The method of any one of claims 76-89, wherein no more than 1 droplet per 10⁵ droplets contain the amplified nucleic acids.

91. The method of any one of claims 76-90, wherein no more than 1 droplet per 10⁶ droplets contain the amplified nucleic acids.
